SEQUENCE LISTING (15 sequences)

SEQ ID NO: 1 - protein, 14 aa, Listeria monocytogenes
KTEEQPSEVNTGPR

SEQ ID NO: 2 - protein, 28 aa, Listeria monocytogenes
KESVVDASESDLDSSMQSADESTPQPLK

SEQ ID NO: 3 - protein, 20 aa, Listeria monocytogenes
KSEEVNASDFPPPPTDEELR

SEQ ID NO: 4 - protein, 33 aa, Listeria monocytogenes
RGGIPTSEEFSSLNSGDFTDDENSETTEEEIDR

SEQ ID NO: 5 - protein, 17 aa, Streptococcus pyogenes
KQNTASTETTTTNEQPK

SEQ ID NO: 6 - protein, 17 aa, Streptococcus equisimilis
KQNTANTETTTTNEQPK

SEQ ID NO: 7 - protein, 633 aa, Listeria monocytogenes
MRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSEEFSSLNSGDFTDDENSETTEEEIDRLADLRDRGTGKHSRNAGFLPLNPFISSPVPSLTPKVPKISAPALISDITKKAPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 8 - protein, 639 aa, Listeria monocytogenes
MGLNRFMRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSEEFSSLNSGDFTDDENSETTEEEIDRLADLRDRGTGKHSRNAGFLPLNPFISSPVPSLTPKVPKISAPALISDITKKAPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 9 - protein, 390 aa, Artificial Sequence
  Truncated ActA
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 10 - protein, 100 aa, Artificial Sequence
  Truncated ActA
MGLNRFMRAMMVVFITANCITINPDIIFAATDSEDSSLNTDEWEEEKTEEQPSEVNTGPRYETAREVSSRDIKELEKSNKVRNTNKADLIAMLKEKAEKG

SEQ ID NO: 11 - protein, 93 aa, Artificial Sequence
  Truncated ActA
ATDSEDSSLNTDEWEEEKTEEQPSEVNTGPRYETAREVSSRDIEELEKSNKVKNTNKADLIAMLKAKAEKGPNNNNNNGEQTGNVAINEEASG

SEQ ID NO: 12 - protein, 200 aa, Artificial Sequence
  Truncated ActA
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 13 - protein, 303 aa, Artificial Sequence
  Truncated ActA
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSS

SEQ ID NO: 14 - protein, 370 aa, Artificial Sequence
  Truncated ActA
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSE

SEQ ID NO: 15 - DNA, 1170 bp, Artificial Sequence
  Truncated ActA
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

(19) United States
(12) Patent Application Publication
(10) Pub. No.: US 2018/0104284 A1
(43) Pub. Date: Apr. 19, 2018

(54) Immunogenic Listeria-Based Compositions Comprising Truncated Acta-Antigen Fusions And Methods Of Use Thereof

(71) Applicant: Advaxis, Inc., Princeton, NJ (US)

(72) Inventors: Anu Wallecha, Yardley, PA (US); Poonam Molli, North Brunswick, NJ (US); Robert Petit, Newtown, PA (US)

(21) Appl. No.: 15/573,382

(22) PCT Filed: May 12, 2016
(86) PCT No.: PCT/US16/32182

§ 371 Date: Nov. 10, 2017

Related U.S. Application Data
(60) Provisional application No. 62/160,764, filed on May 13, 2015.

(51) Int. Cl. A61K 35/74; A61K 39/00; C07K 14/195; C12N 1/36; C07K 16/12

(52) U.S. Cl. CPC A61K 35/74; A61K 39/0011; C07K 14/195; C12N 1/36; C07K 2319/33; A61K 2039/523; A61K 2039/6068; C07K 2317/732; C07K 16/1296

(57) ABSTRACT

The present invention relates to compositions comprising a recombinant attenuated Listeria strain expressing a truncated ActA and fusion proteins thereof and methods of using the same for inducing anti-disease immune responses, and treatment of the same, including a tumor growth or cancer. In particular, the invention relates to the treatment of a tumor growth or cancer using a live attenuated recombinant Listeria strain that expresses a fusion protein of a truncated ActA fused to an antigen.

### CROSS REFERENCE TO RELATED APPLICATIONS

This application claims the benefit of U.S. Provisional Application No. 62/160,764, filed May 13, 2015, the entire contents of which are incorporated herein by reference.

### REFERENCE TO A SEQUENCE LISTING SUBMITTED AS A TEXT FILE VIA EFS WEB

The Sequence Listing written in file 479238SEQLIST.txt is 27.0 kb, was created on May 12, 2016, and is hereby incorporated by reference.

### FIELD OF INVENTION

The present invention relates to compositions comprising a recombinant attenuated Listeria strain expressing a truncated ActA and fusion proteins thereof and methods of using the same for inducing anti-disease immune responses, and treatment of the same, including a tumor growth or cancer. In particular, the invention relates to the treatment of a tumor growth or cancer using a live attenuated recombinant Listeria strain that expresses a fusion protein of a truncated ActA fused to an antigen.

### BACKGROUND OF THE INVENTION

Listeria monocytogenes (L. monocytogenes or Lm) is an intracellular pathogen that primarily infects antigen presenting cells and has adapted for life in the cytoplasm of these cells. Host cells, such as macrophages, actively phagocytose L. monocytogenes and the majority of the bacteria are degraded in the phagolysosome. Some of the bacteria escape into the host cytosol by perforating the phagosomal membrane through the action of a hemolysin, listeriolysin O (LLO). Once in the cytosol, L. monocytogenes can polymerize the host actin and pass directly from cell to cell further evading the host immune system and resulting in a negligible antibody response to L. monocytogenes. Since L. monocytogenes has access to both phagosomal and cytosolic compartments, antigens delivered by Lm can be presented in the context of both MHC I and II molecules, resulting in strong but preferentially cellular immune responses.

PEST sequences in eukaryotic proteins have long been identified. It has been taught that proteins containing amino acid sequences that are rich in prolines (P), glutamic acids (E), serines (S) and threonines (T), generally, but not always, flanked by clusters containing several positively charged amino acids, have rapid intracellular half-lives (Rogers et al., 1986, Science 234:364-369). Further, it has been shown that these sequences target the protein to the ubiquitin-proteosome pathway for degradation (Rechsteiner and Rogers TIBS 1996 21:267-271). This pathway is also used by eukaryotic cells to generate immunogenic peptides that bind to MHC class I and it has been hypothesized that PEST sequences are abundant among eukaryotic proteins that give rise to immunogenic peptides (Realini et al. FEBS Lett. 1994 348:109-113). Prokaryotic proteins do not normally contain PEST sequences because they do not have this enzymatic pathway. However, a PEST-like sequence rich in the amino acids proline (P), glutamic acid (E), serine (S) and threonine (T) was recently identified at the amino terminus of LLO and demonstrated to be essential for L. monocytogenes pathogenicity (Decatur, A. L. and Portnoy, D. A. Science 2000 290:992-995). Decatur and Portnoy teach that the presence of this PEST-like sequence in LLO targets the protein for destruction by proteolytic machinery of the host cell so that once the LLO has served its function and facilitated the escape of L. monocytogenes from the phagolysosomal vacuole, it is destroyed before it can damage the cells.

A Listerial protein, ActA, comprises PEST and PEST-like sequences. ActA is a surface-associated protein, and acts as a scaffold in infected host cells to facilitate the polymerization, assembly and activation of host actin polymers in order to propel the Listeria organism through the cytoplasm. Shortly after entry into the mammalian cell cytosol, L. monocytogenes induces the polymerization of host actin filaments and uses the force generated by actin polymerization to move, first intracellularly and then from cell to cell. A single bacterial protein, ActA is responsible for mediating actin nucleation and actin-based motility. The ActA protein provides multiple binding sites for host cytoskeletal components, thereby acting as a scaffold to assemble the cellular actin polymerization machinery. The NH₂ terminus of ActA binds to monomeric actin and acts as a constitutively active nucleation promoting factor by stimulating the intrinsic actin nucleation activity. ActA and hly are both members of the 10-kb gene cluster regulated by the transcriptional activator PrfA, and is upregulated approximately 226-fold in the mammalian cytosol.

There exists a long-felt need to develop compositions and methods to enhance the immunogenicity of antigens, especially antigens useful in the prevention and treatment of tumors and intracellular pathogens. The present invention meets this need by providing an immunogenic truncated ActA protein to which a heterologous antigen can be fused to in order to enhance the immunogenicity of the heterologous antigen.

### SUMMARY OF THE INVENTION

In one aspect, the invention provided herein relates to a recombinant Listeria strain comprising a nucleic acid molecule comprising a first open reading frame encoding a recombinant polypeptide, said polypeptide comprising a truncated ActA protein fused to an antigen.

In a related aspect, the invention provided herein relates to a recombinant Listeria strain comprising a nucleic acid molecule comprising a first open reading frame encoding a recombinant polypeptide, said polypeptide comprising a truncated ActA protein, wherein said nucleic acid molecule comprises a second open reading frame encoding a metabolic enzyme that complements a mutation, deletion, or inactivation in a gene encoding a metabolic enzyme in said Listeria strain's chromosome. In another aspect, the metabolic enzyme is an alanine racemase enzyme or a D-amino acid transferase enzyme. In another aspect, said recombinant Listeria comprises a mutation in the actA virulence gene. In another aspect, said recombinant attenuated Listeria is a Listeria monocytogenes.

In another related aspect, the invention provided herein relates to a pharmaceutical composition comprising a recombinant Listeria strain provided herein, or a recombinant polypeptide provided herein, or a recombinant nucleic acid molecule provided herein, and a pharmaceutically acceptable carrier.

In another related aspect, the invention provided herein relates to a method of inducing an anti-disease immune response in a subject, the method comprising the step of administering a composition comprising a recombinant Listeria strain, said Listeria strain comprising a recombinant nucleic acid molecule comprising a first open reading frame encoding a recombinant polypeptide, said recombinant polypeptide comprising a truncated ActA fused to an antigen, thereby inducing an anti-disease immune response in a subject. In another embodiment, said nucleic acid molecule comprises a second open reading frame encoding a metabolic enzyme that complements a mutation, deletion, or inactivation in a gene encoding a metabolic enzyme in said Listeria strain's chromosome.

In another related aspect, the invention provided herein relates to a method of delaying metastatic disease in a subject having a disease, said method comprising the step of administering a composition comprising a recombinant Listeria strain, said Listeria strain comprising a recombinant nucleic acid comprising a first open reading frame encoding a recombinant polypeptide, said recombinant polypeptide comprising a truncated ActA fused to an antigen, thereby inducing an anti-disease immune response in a subject. In one aspect, the disease is a tumor growth or cancer.

In another related aspect, the invention provided herein relates to a method of breaking tolerance to a self-antigen in a subject having a disease, said method comprising a composition comprising a recombinant Listeria provided herein. In one aspect, the disease is a tumor growth or cancer.

In another related aspect, the invention provided herein relates to a method of delaying the onset of or preventing a disease, the method comprising a composition comprising a recombinant Listeria provided herein. In one aspect, the disease is a tumor growth or cancer.

In another related aspect, the invention provided herein relates to a method of treating a disease in a subject, the method comprising a composition comprising a recombinant Listeria provided herein. In one aspect, the disease is a tumor growth or cancer.

In another related aspect, the invention provided herein relates to a kit comprising a pharmaceutical composition, recombinant Listeria, recombinant peptide, or recombinant nucleic acid provided herein.

In another related aspect, a subject is a human. In another aspect, administering a composition comprising said recombinant attenuated Listeria prevents escape mutations within a tumor or cancer, results in progression free survival, inhibiting tumor growth, inducing cancer regression, extension of progression free survival (PFS), increasing time to disease progression, or any combination thereof.

In another related aspect, provided herein is a method of inducing an anti-tumor immune response, said method comprising the step of administering a combination therapy comprising a composition comprising an immunosuppressive antagonist and a composition comprising a recombinant Listeria provided herein.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1. Schematic map of the plasmid pAdv142. The plasmid contains both Listeria and E. coli origin of replication (A). The antigen expression cassette consists of hly promoter, ORF for truncated LLO and human PSA gene (klk3). The western blot from LmddA-LLO-PSA supernatants shows the expression of LLO-PSA fusion protein using anti-PSA and anti-LLO antibody (B). A schematic representation showing the cloning of the different ActA PEST regions in the plasmid backbone pAdv142 to create plasmids pAdv211, pAdv223 and pAdv224 is shown in (C). This schematic shows different ActA coding regions were cloned in frame with Listeriolysin O signal sequence in the backbone plasmid pAdv142, restricted with XbaI and XhoI (C).

FIG. 2. (A) Tumor regression study using TPSA23 as transplantable tumor model. Three groups of eight mice were implanted with 1×10⁶ tumor cells on day 0 and were treated on day 6, 13 and 20 with 10⁸ CFU of different therapies: LmddA142, LmddA211, LmddA223 and LmddA224. Naïve mice did not receive any treatment. Tumors were monitored weekly and mice were sacrificed if the average tumor diameter was 14-18 mm. Each symbol in the graph represents the tumors size of an individual mouse. The experiment was repeated twice and similar results were obtained. (B) The percentage survival of the naïve mice and immunized mice at different days of the experiment.

FIG. 3. PSA specific immune responses were examined by tetramer staining (A) and intracellular cytokine staining for IFN-γ (B). Mice were immunized three times at weekly intervals with 10⁸ CFU of different therapies: LmddA142 (ADXS31-142), LmddA211, LmddA223 and LmddA224. For immune assays, spleens were harvested on day 6 after the second boost. Spleens from 2 mice/group were pooled for this experiment. (A) PSA specific T cells in the spleen of naïve, LmddA142, LmddA211, LmddA223 and LmddA224 immunized mice were detected using PSA-epitope specific tetramer staining. Cells were stained with mouse anti-CD8 (FITC), anti-CD3 (Percp-Cy5.5), anti-CD62L (APC) and PSA tetramer-PE and analyzed by FACS Calibur. (B) Intracellular cytokine staining to detect the percentage of IFN-γ secreting CD8+ CD62Llow cells in the naïve and immunized mice after stimulation with 1 μM of PSA specific, H-2Db peptide (HCIRNKSVIL) for 5 h.

FIG. 4. TPSA23, tumor model was used to study immune response generation in C57BL6 mice by using ActA/PEST2 (LA229) fused PSA and tLLO fused PSA. Four groups of five mice were implanted with 1×10⁶ tumor cells on day 0 and were treated on day 6 and 14 with 10⁸ CFU of different therapies: LmddA274, LmddA142 (ADXS31-142) and LmddA211. Naïve mice did not receive any treatment. On Day 6 post last immunization, spleen and tumor was collected from each mouse. A) Table shows the tumor volume on day 13 post immunization. PSA specific immune responses were examined by pentamer staining in spleen (B) and in tumor (C). For immune assays, spleens from 2 mice/group or 3 mice/group were pooled and tumors from 5 mice/group was pooled. Cells were stained with mouse anti-CD8 (FITC), anti-CD3 (Percp-Cy5.5), anti-CD62L (APC) and PSA Pentamer-PE and analyzed by FACS Calibur.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Abbreviations. Throughout the detailed description and examples of the invention the following abbreviations will be used:

APC antigen presenting cell

BID One dose twice daily

CFU Colony-forming units

CHO Chinese hamster ovary

DFS Disease free survival

FFPE formalin-fixed, paraffin-embedded

IHC Immunohistochemistry or immunohistochemical

Lm Listeria monocytogenes

NCBI National Center for Biotechnology Information

NCI National Cancer Institute

OR Overall response

ORF Open reading frame

OS Overall survival

PCR Polymerase chain reaction

PD Progressive disease

PFS Progression free survival

PR Partial response

Q2W One dose every two weeks

Q3W One dose every three weeks

Q4W One dose every four weeks

QD One dose per day

RECIST Response Evaluation Criteria in Solid Tumors

SD Stable disease

SDS-PAGE Sodium dodecyl sulfate-Polyacrylamide gel electrophoresis

TILs Tumor infiltrating lymphocytes

### I. Recombinant Listeria Strains and Compositions Comprising the Same

In one embodiment, provided herein is a recombinant attenuated Listeria strain comprising a nucleic acid molecule comprising a first open reading frame encoding a recombinant polypeptide, wherein the recombinant polypeptide comprises an antigen or immunogenic fragment thereof fused to a truncated ActA protein.

In another embodiment, a truncated ActA protein is fragment of an ActA protein. In another embodiment, the truncated ActA protein is an N-terminal fragment of an ActA protein. In another embodiment, a nucleic acid molecule provided herein further comprises a second open reading frame encoding a metabolic enzyme, wherein the metabolic enzyme complements a mutation, deletion or inactivation in the chromosome of the recombinant Listeria strain. In another embodiment, the metabolic enzyme complements a deletion in the chromosome of the recombinant Listeria strain. In another embodiment, the metabolic enzyme complements a genomic mutation, deletion or inactivation in a gene encoding a metabolic enzyme in the recombinant Listeria strain. In another embodiment, the nucleic acid molecule further comprises a third open reading frame encoding a metabolic enzyme, wherein the metabolic enzyme complements a mutation, deletion or inactivation in the chromosome of the recombinant Listeria strain. In another embodiment, the metabolic enzyme complements a deletion in the chromosome of the recombinant Listeria strain. In another embodiment, the metabolic enzyme complements a genomic mutation, deletion or inactivation in a gene encoding a metabolic enzyme in the recombinant Listeria strain. In another embodiment, the metabolic enzyme is an alanine racemase enzyme or a D-amino acid transferase enzyme. In another aspect, said recombinant Listeria comprises a mutation in the actA virulence gene. In another aspect, said recombinant attenuated Listeria is a Listeria monocytogenes.

In one embodiment, the terms “recombinant Listeria” and “live-attenuated Listeria” are used interchangeably here and refer to a Listeria comprising at least one attenuating mutation, deletion or inactivation that expresses a fusion protein of an antigen fused to a truncated ActA embodied herein.

The terms “nucleic acids,” “nucleotide,” “nucleic acid molecule,” “oligonucleotide,” or “nucleotide molecule” are used interchangeably herein and may encompass a string of at least two base-sugar-phosphate combinations. The term includes, in one embodiment, DNA and RNA. It will also be appreciated by a skilled artisan that the term “nucleotide” may encompass the monomeric units of nucleic acid polymers. For example, RNA may be in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA may be in form of plasmid DNA, viral DNA, linear DNA, or chromosomal DNA or derivatives of these groups. In addition, these forms of DNA and RNA may be single, double, triple, or quadruple stranded. The term may also encompass artificial nucleic acids that may contain other types of backbones but the same bases. The use of phosphothiorate nucleic acids and PNA are known to those skilled in the art, and are described in, for example, Neilsen P E, Curr Opin Struct Biol 9:353-57; and Raz N K et al Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed.

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” may include both D- and L-amino acids.

It will be appreciated by a skilled artisan that the term “open reading frame” or “ORF” may encompass a portion of an organism's genome which contains a sequence of bases that could potentially encode a protein. In another embodiment, the start and stop ends of the ORF are not equivalent to the ends of the mRNA, but they are usually contained within the mRNA. In one embodiment, ORFs are located between the start-code sequence (initiation codon) and the stop-codon sequence (termination codon) of a gene. Thus, in one embodiment, a nucleic acid molecule operably integrated into a genome as an open reading frame with an endogenous polypeptide is a nucleic acid molecule that has integrated into a genome in the same open reading frame as an endogenous polypeptide.

It will be appreciated by a skilled artisan that the term “endogenous” may encompass an item that has developed or originated within the reference organism or arisen from causes within the reference organism. For example, endogenous refers to native.

It will be appreciated by a skilled artisan that the term “fragment” may encompass a protein or polypeptide that is shorter or comprises fewer amino acids than the full length protein or polypeptide. In one embodiment, a fragment is an N-terminal fragment. In another embodiment, a fragment is a C-terminal fragment. In yet another embodiment, a fragment is an intrasequential section of the protein or peptide. It will be understood by a skilled artisan that a fragment as provided herein is a functional fragment, which may encompass an immunogenic fragment. In one embodiment, a fragment has more than 5 amino acids. In another embodiment, a fragment has 10-20 amino acids, 20-50 amino acids, 50-100 amino acids, 100-200 amino acids, 200-350 amino acids, or 350-500 amino acids.

In an alternate embodiment, the term “fragment” when in reference to a nucleic acid refers to a nucleic acid sequence that is shorter or comprises fewer nucleotides than the full length nucleic acid. In one embodiment, a fragment is a 5′-terminal fragment. In another embodiment, a fragment is a 3′-terminal fragment. In yet another embodiment, a fragment encodes an intrasequential section of the protein. In one embodiment, a fragment has more than 5 nucleotides. In another embodiment, a fragment has 10-20 nucleotides, 20-50 nucleotides, 50-100 nucleotides, 100-200 nucleotides, 200-350 nucleotides, 350-500 or 500-1000 nucleotides. It will be appreciated by a skilled artisan that the term “functional” within the meaning of the invention, may encompass the innate ability of a protein, peptide, nucleic acid, fragment or a variant thereof to exhibit a biological activity. Such a biological activity may encompass having the potential to elicit an immune response when used as provided herein, an illustration of which may be to be used as part of a fusion protein). Such a biological function may encompass its binding property to an interaction partner, e.g., a membrane-associated receptor, or its trimerization property. In the case of functional fragments and the functional variants of the invention, these biological functions may in fact be changed, e.g., with respect to their specificity or selectivity, but with retention of the basic biological function.

It will be appreciated by a skilled artisan that the term “functional fragment” may encompass an immunogenic fragment that is capable of eliciting an immune response when administered to a subject alone or as part of a pharmaceutical composition comprising a recombinant Listeria strain expressing said immunogenic fragment. In another embodiment, a functional fragment has biological activity as will be understood by a skilled artisan and as further provided herein.

It will be appreciated by a skilled artisan that the term “fused” may encompass an operable linkage by covalent bonding. In one embodiment, the term encompasses recombinant fusion (of nucleic acid sequences or open reading frames thereof). In another embodiment, the term encompasses chemical conjugation.

In another embodiment, a PEST AA sequence comprises a truncated ActA sequence. In another embodiment, a truncated ActA sequence comprises a PEST sequence. In another embodiment, PEST AA sequence comprises an ActA fragment sequence.

It will be appreciated by the skilled artisan that the terms “PEST amino acid sequence,” “PEST AA sequence,” “PEST sequence-containing polypeptide,” “PEST sequence-containing protein,” or “PEST-containing peptide or polypeptide” are used interchangeably herein and may encompass a PEST sequence peptide, which may encompass a fragment of an ActA protein. PEST sequence peptides are known in the art and are described in U.S. Pat. Ser. No. 7,635,479, in U.S. Pat. Ser. No. 7,665,238 and in US Patent Publication Serial No. 2014/0186387, all of which are hereby incorporated in their entirety herein.

In one embodiment, fusion of an antigen to a truncated ActA comprising a PEST sequence of Listeria monocytogenes enhances cell mediated and anti-tumor immunity of the antigen. Thus, fusion of an antigen to PEST-amino acid sequences from other prokaryotic organisms (including but not limited to, other Listeria species) would be expected to have similar effect. In another embodiment, the PEST sequence is embedded within the antigenic protein. Thus, “fusion” refers to an antigenic protein comprising both the antigen and the PEST amino acid sequence either linked at one end of the antigen or embedded within the antigen. PEST sequences derived from other prokaryotic organisms will also enhance immunogenicity of the antigen. In another embodiment, a PEST sequence of prokaryotic organisms can be identified routinely in accordance with methods such as described by Rechsteiner and Roberts (TBS 21:267-271, 1996) for L. monocytogenes. Alternatively, PEST amino acid sequences from other prokaryotic organisms can also be identified based by this method. Other prokaryotic organisms wherein PEST amino acid sequences would be expected include, but are not limited to, other Listeria species. For example, the L. monocytogenes protein ActA contains four such sequences. These are KTEEQPSEVNTGPR (SEQ ID NO: 1), KESVVDASESDLDSSMQSADESTPQPLK (SEQ ID NO: 2), KSEEVNASDFPPPPTDEELR (SEQ ID NO: 3), and RGGIPTSEEFSSLNSGDFTDDENSETTEEEIDR (SEQ ID NO: 4). Also Streptolysin O from Streptococcus sp. contains a PEST sequence. For example, Streptococcus pyogenes Streptolysin O comprises the PEST sequence KQNTASTETTTTNEQPK (SEQ ID NO: 5) at amino acids 35-51 and Streptococcus equisimilis Streptolysin O comprises the PEST sequence KQNTANTETTTTNEQPK (SEQ ID NO: 6) at amino acids 38-54. Further, it is believed that the PEST sequence can be embedded within the antigenic protein. Thus, it will be appreciated by a skilled artisan that a “fusion” when in relation to PEST sequence fusions, may encompass an operable linkage of an antigenic protein or fragment thereof to a PEST amino acid sequence linked at one end of the antigen. Alternatively, the PEST amino acid sequence may be embedded within the antigen.

The terms “antigen,” “antigenic polypeptide,” “antigen fragment,” are used interchangeably herein and, as will be appreciated by a skilled artisan, may encompass polypeptides, or peptides (including recombinant peptides) that are loaded onto and presented on MHC class I and/or class II molecules on a host's cell's surface and can be recognized or detected by an immune cell of the host, thereby leading to the mounting of an immune response against the polypeptide, peptide or cell presenting the same. Similarly, the immune response may also extend to other cells within the host, including diseased cells such as tumor or cancer cells that express the same polypeptides or peptides.

It will also be appreciated by a skilled artisan that the terms “antigenic portion thereof”, “a fragment thereof” and “immunogenic portion thereof” in regard to a protein, peptide or polypeptide are used interchangeably herein and may encompass a protein, polypeptide, peptide, including recombinant forms thereof comprising a domain or segment that leads to the mounting of an immune response when present in, or, in some embodiments, detected by, a host, either alone, or in the context of a fusion protein, as described herein.

In one embodiment, an antigen may be foreign, that is, heterologous to the host and is referred to as a “heretologous antigen” herein. In another embodiment, the antigen is a self-antigen, which is an antigen that is present in the host but the host does not elicit an immune response against it because of immunologic tolerance. It will be appreciated by a skilled artisan that a heterologous antigen as well as a self-antigen may encompass a tumor antigen, a tumor-associated antigen or an angiogenic antigen. In addition, a heterologous antigen may encompass an infectious disease antigen.

In one embodiment, the tumor-associated antigen is selected from HPV-E7, HPV-E6, Her-2, NY-ESO-1, SCCE, WT-1, Proteinase 3, HMW-MAA, a VEGFR-2 fragment, survivin, a B-cell receptor antigen, Tyrosinase related protein 2, or a PSA (prostate-specific antigen) or a combination thereof. In another embodiment, the antigen is an infectious disease antigen such as is HIV-1 Gag, a MAGE (Melanoma-Associated Antigen E) protein, e.g. MAGE 1, MAGE 2, MAGE 3, MAGE 4, a tyrosinase; a mutant ras protein; a mutant p53 protein; p97 melanoma antigen, a ras peptide or p53 peptide associated with advanced cancers; the HPV 16/18 antigens associated with cervical cancers, KLH antigen associated with breast carcinoma, CEA (carcinoembryonic antigen) associated with colorectal cancer, gp100, a MART1 antigen associated with melanoma, a telomerase (TERT), SCCE, CEA, LMP-1, p53, carboxic anhydrase IX (CAIX). PSMA, prostate stem cell antigen (PSCA), or WT-1.

In one embodiment an HPV antigen such as an E6 or E7 antigen provided herein is selected from an HPV 16 strain, an HPV-18 strain, an HPV-31 strain, an HPV-35 strain, an HPV-39 strain, an HPV-45 strain, an HPV-52 strain, or an HPV-58 strain. In another embodiment, the HPV antigen is selected from a high-risk HPV strain. In another embodiment, the HPV strain is a mucosal HPV type.

In one embodiment, an HPV-16 E6 and E7 are utilized instead of or in combination with an HPV-18 E6 and E7. In such an embodiment, the recombinant Listeria may express the HPV-16 E6 and E7 from the chromosome and the HPV-18 E6 and E7 from a plasmid, or vice versa. In another embodiment, the HPV-16 E6 and E7 antigens and the HPV-18 E6 and E7 antigens are expressed from a plasmid present in a recombinant Listeria provided herein. In another embodiment, the HPV-16 E6 and E7 antigens and the HPV-18 E6 and E7 antigens are expressed from the chromosome of a recombinant Listeria provided herein. In another embodiment, the HPV-16 E6 and E7 antigens and the HPV-18 E6 and E7 antigens are expressed in any combination of the above embodiments, including where each E6 and E7 antigen from each HPV strain is expressed from either the plasmid or the chromosome.

In one embodiment, the antigen is a chimeric Her2 antigen described in U.S. patent application Ser. No. 12/945,386, which is hereby incorporated by reference herein in its entirety.

In other embodiments, the antigen is associated with one of the following diseases; cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough, yellow fever, the immunogens and antigens from Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, non-Hodgkin's lymphoma, malarial circumsporozite protein, microbial antigens, viral antigens, autoantigens, and listeriosis.

In another embodiment, the tumor-associated antigen provided herein is an angiogenic antigen which is expressed on both activated pericytes and pericytes in tumor angiogeneic vasculature, which is associated with neovascularization in vivo. Angiogenic antigens are known in the art see for example WO2010/102140, which is incorporated by reference herein. For example, an angiogenic factor may be selected from; Angiopoietin-1 (Ang1), Angiopoietin 3, Angiopoietin 4, Angiopoietin 6; Del-1; Fibroblast growth factors: acidic (aFGF) and basic (bFGF); Follistatin; Granulocyte colony-stimulating factor (G-CSF); Hepatocyte growth factor (HGF)/scatter factor (SF); Interleukin-8 (IL-8); Leptin; Midkine; Placental growth factor; Platelet-derived endothelial cell growth factor (PD-ECGF); Platelet-derived growth factor-BB (PDGF-BB); Pleiotrophin (PTN); Progranulin; Proliferin; survivin; Transforming growth factor-alpha (TGF-alpha); Transforming growth factor-beta (TGF-beta); Tumor necrosis factor-alpha (TNF-alpha); Vascular endothelial growth factor (VEGF)/vascular permeability factor (VPF). In another embodiment, an angiogenic factor is an angiogenic protein. In one embodiment, a growth factor is an angiogenic protein. In one embodiment, an angiogenic protein for use in the compositions and methods of the present invention is Fibroblast growth factors (FGF); VEGF; VEGFR and Neuropilin 1 (NRP-1); Tie2; Platelet-derived growth factor (PDGF; BB-homodimer) and PDGFR; Transforming growth factor-beta (TGF-β), endoglin and TGF-β receptors; monocyte chemotactic protein-1 (MCP-1); Integrins αVPβ3, αVPβ5 and α5β1; VE-cadherin and CD31; ephrin; plasminogen activators; plasminogen activator inhibitor-1; Nitric oxide synthase (NOS) and COX-2; AC133; or Id1/Id3, a TGFbeta co-receptor or endoglin (which is also known as CD105; EDG; HHT1; ORW; or ORW1).

It will be appreciated by a skilled artisan that the compositions provided herein when administered to a subject generate effector T cells that are able to infiltrate the tumor, destroy tumor cells and eradicate the disease. In one embodiment, naturally occurring tumor infiltrating lymphocytes (TILs) are associated with better prognosis in several tumors, such as colon, ovarian and melanoma. In colon cancer, tumors without signs of micrometastasis have an increased infiltration of immune cells and a Th1 expression profile, which correlate with an improved survival of patients. Moreover, the infiltration of the tumor by T cells has been associated with success of immunotherapeutic approaches in both pre-clinical and human trials. In one embodiment, the infiltration of lymphocytes into the tumor site is dependent on the up-regulation of adhesion molecules in the endothelial cells of the tumor vasculature, generally by proinflammatory cytokines, such as IFN-γ, TNF-α and IL-1. Several adhesion molecules have been implicated in the process of lymphocyte infiltration into tumors, including intercellular adhesion molecule 1 (ICAM-1), vascular endothelial cell adhesion molecule 1 (V-CAM-1), vascular adhesion protein 1 (VAP-1) and E-selectin. However, these cell-adhesion molecules are commonly down-regulated in the tumor vasculature. Thus, in one embodiment, cancer vaccines as provided herein increase TILs, up-regulate adhesion molecules (in one embodiment, ICAM-1, V-CAM-1, VAP-1, E-selectin, or a combination thereof), up-regulate pro-inflammatory cytokines (in one embodiment, IFN-γ, TNF-α, IL-1, or a combination thereof), or a combination thereof.

In one embodiment, compositions provided herein induce a strong innate stimulation of interferon-gamma, which in one embodiment has anti-angiogenic properties. In one embodiment, a Listeria of the present invention induces a strong innate stimulation of interferon-gamma, which in one embodiment, has anti-angiogenic properties (Dominiecki et al., Cancer Immunol Immunother. 2005 May; 54(5):477-88. Epub 2004 Oct 6., incorporated herein by reference in its entirety; Beatty and Paterson, J Immunol. 2001 Feb. 15; 166(4):2276-82, incorporated herein by reference in its entirety). In one embodiment, anti-angiogenic properties of Listeria are mediated by CD4⁺ T cells (Beatty and Paterson, 2001). In another embodiment, anti-angiogenic properties of Listeria are mediated by CD8⁺ T cells. In another embodiment, IFN-gamma secretion as a result of Listeria vaccination is mediated by NK cells, NKT cells, Th1 CD4⁺ T cells, TC1 CD8⁺ T cells, or a combination thereof.

In another embodiment, compositions provided herein induce production of one or more anti-angiogenic proteins or factors. In one embodiment, the anti-angiogenic protein is IFN-gamma. In another embodiment, the anti-angiogenic protein is pigment epithelium-derived factor (PEDF); angiostatin; endostatin; fms-like tyrosine kinase (sFlt)-1; or soluble endoglin (sEng). In one embodiment, a Listeria of the present invention is involved in the release of anti-angiogenic factors, and, therefore, in one embodiment, has a therapeutic role in addition to its role as a vector for introducing an antigen to a subject. Each Listeria strain and type thereof represents a separate embodiment of the present invention.

In other embodiments, the antigen is derived from a fungal pathogen, bacteria, parasite, helminth, or viruses. An illustrative antigen may be selected from tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, human papilloma virus antigens E1 and E2 from type HPV-16, -18, -31, -33, -35 or -45 human papilloma viruses.

It will be appreciated by a skilled artisan that the term “immunogenicity” or “immunogenic” may encompass the innate ability of a protein, peptide, nucleic acid, antigen or organism to elicit an immune response in an animal when the protein, peptide, nucleic acid, antigen or organism is administered to the animal. Thus, “enhancing the immunogenicity” in one embodiment, refers to increasing the ability of a protein, peptide, nucleic acid, antigen or organism to elicit an immune response in an animal when the protein, peptide, nucleic acid, antigen or organism is administered to an animal. The increased ability of a protein, peptide, nucleic acid, antigen or organism to elicit an immune response can be measured by a greater number of antibodies to a protein, peptide, nucleic acid, antigen or organism, a greater diversity of antibodies to an antigen or organism, a greater number of T-cells specific for a protein, peptide, nucleic acid, antigen or organism, a greater cytotoxic or helper T-cell response to a protein, peptide, nucleic acid, antigen or organism, and the like.

In another embodiment, a nucleic acid molecule provided herein is expressed from an episomal or plasmid vector, with a nucleic acid sequence encoding a truncated ActA protein. In another embodiment, the plasmid is stably maintained in the recombinant Listeria vaccine strain in the absence of antibiotic selection. In another embodiment, the plasmid does not confer antibiotic resistance upon the recombinant Listeria.

It will be appreciated by a skilled artisan that the term “vector” may encompass a extrachromosomal plasmid capable of replicating in the cytoplasm of a Listeria host or an integration plasmid capable of being transformed into a Listeria host and being incorporated in the Listeria's chromosome in a manner that allows expression of the genes comprised by the plasmid. In another embodiment, an integration vector is a site-specific integration vector.

The skilled artisan, when equipped with the present disclosure, will readily understand that different transcriptional promoters, terminators, carrier vectors or specific gene sequences (e.g. those in commercially available cloning vectors) can be used successfully in the methods and compositions provided herein. As is contemplated in the present invention, these functionalities are provided in, for example, the commercially available vectors known as the pUC series. In one embodiment, non-essential DNA sequences (e.g. antibiotic resistance genes) are removed. In another embodiment, a commercially available plasmid is used in the present invention. Such plasmids are available from a variety of sources, for example, Invitrogen (La Jolla, Calif.), Stratagene (La Jolla, Calif.), Clontech (Palo Alto, Calif.), or can be constructed using methods well known in the art. Another embodiment is a plasmid such as pCR2.1 (Invitrogen, La Jolla, Calif.), which is a prokaryotic expression vector with a prokaryotic origin of replication and promoter/regulatory elements to facilitate expression in a prokaryotic organism. In another embodiment, extraneous nucleotide sequences are removed to decrease the size of the plasmid and increase the size of the cassette that can be placed therein. Such methods are well known in the art, and are described in, for example, Sambrook et al. (1989, Molecular Cloning: A Laboratory Manual, Cold Spring Harbor Laboratory Press, New York) and Ausubei et al. (1997, Current Protocols in Molecular Biology, Green & Wiley, New York).

In one embodiment, an ActA protein provided herein comprises a sequence set forth in SEQ ID NO: 7:

(SEQ ID NO: 7)
MRAMMVVFIT ANCITINPDI IFAATDSEDS SLNTDEWEEE
KTEEQPSEVN TGPRYETARE VSSRDIEELE KSNKVKNTNK
ADLIAMLKAK AEKGPNNNNN NGEQTGNVAI NEEASGVDRP
TLQVERRHPG LSSDSAAEIK KRRKAIASSD SELESLTYPD
KPTKANKRKV AKESVVDASE SDLDSSMQSA DESTPQPLKA
NQKPFFPKVF KKIKDAGKWV RDKIDENPEV KKAIVDKSAG
LIDQLLTKKK SEEVNASDFP PPPTDEELRL ALPETPMLLG
FNAPTPSEPS SFEFPPPPTD EELRLALPET PMLLGFNAPA
TSEPSSFEFP PPPTEDELEI MRETAPSLDS SFTSGDLASL
RSAINRHSEN FSDFPLIPTE EELNGRGGRP TSEEFSSLNS
GDFTDDENSE TTEEEIDRLA DLRDRGTGKH SRNAGFLPLN
PFISSPVPSL TPKVPKISAP ALISDITKKA PFKNPSQPLN
VFNKKTTTKT VTKKPTPVKT APKLAELPAT KPQETVLREN
KTPFIEKQAE TNKQSINMPS LPVIQKEATE SDKEEMKPQ
TEEKMVEESE SANNANGKNR SAGIEEGKLI AKSAEDEKAKE
EPGNHTTLILAMLAIGVFSLGAFIKIIQLRKNN.

The first 29 AA of the proprotein corresponding to this sequence are the signal sequence and are cleaved from ActA protein when it is secreted by the bacterium. In one embodiment, an ActA polypeptide or peptide comprises the signal sequence, AA 1-29 of SEQ ID NO: 7 above. In another embodiment, an ActA polypeptide or peptide does not include the signal sequence, AA 1-29 of SEQ ID NO: 7 above.

In another embodiment, an ActA protein is encoded by the sequence set forth in SEQ ID NO: 8

(SEQ ID NO: 8)
M G L N R F M R A M M V V F I T A N C I T I N P D
I I F A A T D S E D S S L N T D E W E E E K T E E
Q P S E V N T G P R Y E T A R E V S S R D I E E L
E K S N K V K N T N K A D L I A M L K A K A E K G
P N N N N N N G E Q T G N V A I N E E A S G V D R
P T L Q V E R R H P G L S S D S A A E I K K R R K
A I A S S D S E L E S L T Y P D K P T K A N K R K
V A K E S V V D A S E S D L D S S M Q S A D E S T
P Q P L K A N Q K P F F P K V F K K I K D A G K W
V R D K I D E N P E V K K A I V D K S A G L I D Q
L L T K K K S E E V N A S D F P P P P T D E E L R
L A L P E T P M L L G F N A P T P S E P S S F E F
P P P P T D E E L R L A L P E T P M L L G F N A P
A T S E P S S F E F P P P P T E D E L E I M R E T
A P S L D S S F T S G D L A S L R S A I N R H S E
N F S D F P L I P T E E E L N G R G G R P T S E E
F S S L N S G D F T D D E N S E T T E E E I D R L
A D L R D R G T G K H S R N A G F L P L N P F I S
S P V P S L T P K V P K I S A P A L I S D I T K K
A P F K N P S Q P L N V F N K K T T T K T V T K K
P T P V K T A P K L A E L P A T K P Q E T V L R E
N K T P F I E K Q A E T N K Q S I N M P S L P V I
Q K E A T E S D K E E M K P Q T E E K M V E E S E
S A N N A N G K N R S A G I E E G K L I A K S A E
D E K A K E E P G N H T T L I L A M L A I G V F S
L G A F I K I I Q L R K N N.

The first 29 AA of the proprotein corresponding to this sequence are the signal sequence and are cleaved from ActA protein when it is secreted by the bacterium. In one embodiment, an ActA polypeptide or peptide comprises the signal sequence, AA 1-29 of SEQ ID NO: 8 above. In another embodiment, an ActA polypeptide or peptide does not include the signal sequence, AA 1-29 of SEQ ID NO: 8 above.

In one embodiment, a truncated ActA protein comprises the sequence set forth in SEQ ID NO: 9

(SEQ ID NO: 9)
MRAMMVVFITANCITINPDIIFAATDSEDSSLNTDEWEEEKTEEQPSEVN
TGPRYETAREVSSRDIKELEKSNKVRNTNKADLIAMLICEKAEKGPNINN
NNSEQTENAAINEEASGADRPAIQVERRHPGLPSDSAAEIKKRRKAIASS
DSELESLTYPDKPTKVNKKKVAKESVADASESDLDSSMQSADESSPQPLK
ANQQPFFPKVFKKIKDAGKWVRDKIDENPEVKKAIVDKSAGLIDQLLTKK
KSEEVNASDFPPPPTDEELRLALPETPMLLGFNAPATSEPSSFEFPPPPT
DEELRLALPETPMLLGFNAPATSEPSSFEFPPPPTEDELEIIRETASSLD
SSFTRGDLASLRNAINRHSQNFSDFPPIPTKEELNGRGGRP.

In another embodiment, a truncated ActA protein comprises the sequence set forth in SEQ ID NO: 10

(SEQ ID NO: 10)
MGLNRFMRAMMVVFITANCITINPDIIFAATDSEDSSLNTDEWEEEKTEE
QPSIKVNTGPRYETAREVSSRDIKELEKSNKVRNTNKADLIAMLKEKAEK
G.

In another embodiment, a truncated ActA protein comprises the sequence set forth in SEQ ID NO: 11

(SEQ ID NO: 11)
A T D S E D S S L N T D E W E E E K T E E Q P S E
V N T G P R Y E T A R E V S S R D I E E L E K S N
K V K N T N K A D L I A M L K A K A E K G P N N N
N N N G E Q T G N V A I N E E A S G.

In another embodiment, a truncated ActA as set forth in SEQ ID NO: 11 is referred to as ActA/PEST1. In another embodiment, a truncated ActA comprises from the first 30 to amino acid 122 of the full length ActA sequence. In another embodiment, SEQ ID NO: 11 comprises from the first 30 to amino acid 122 of the full length ActA sequence. In another embodiment, a truncated ActA comprises from the first 30 to amino acid 122 of SEQ ID NO: 8. In another embodiment, SEQ ID NO: 11 comprises from the first 30 to amino acid 122 of SEQ ID NO: 8.

In another embodiment, a truncated ActA protein comprises the sequence set forth in SEQ TD NO: 12

(SEQ ID NO: 12)
A T D S E D S S L N T D E W E E E K T E E Q P S E
V N T G P R Y E T A R E V S S R D I E E L E K S N
K V K N T N K A D L I A M L K A K A E K G P N N N
N N N G E Q T G N V A I N E E A S G V D R P T L Q
V E R R H P G L S S D S A A E I K K R R K A I A S
S D S E L E S L T Y P D K P T K A N K R K V A K E
S V V D A S E S D L D S S M Q S A D E S T P Q P L
K A N Q K P F F P K V F K K I K D A G K W V R D K.

In another embodiment, a truncated ActA as set forth in SEQ ID NO: 12 is referred to as ActA/PEST2. In another embodiment, a truncated ActA as set forth in SEQ ID NO: 12 is referred to as LA229. In another embodiment, a truncated ActA comprises from amino acid 30 to amino acid 229 of the full length ActA sequence. In another embodiment, SEQ ID NO: 12 comprises from about amino acid 30 to about amino acid 229 of the full length ActA sequence. In another embodiment, a truncated ActA comprises from about amino acid 30 to amino acid 229 of SEQ ID NO: 8. In another embodiment, SEQ ID NO: 12 comprises from amino acid 30 to amino acid 229 of SEQ ID NO: 8.

In another embodiment, a truncated ActA protein comprises the sequence set forth in SEQ ID NO: 13

(SEQ ID NO: 13)
A T D S E D S S L N T D E W E E E K T E E Q P S E
V N T G P R Y E T A R E V S S R D I E E L E K S N
K V K N T N K A D L I A M L K A K A E K G P N N N
N N N G E Q T G N V A I N E E A S G V D R P T L Q
V E R R H P G L S S D S A A E I K K R R K A I A S
S D S E L E S L T Y P D K P T K A N K R K V A K E
S V V D A S E S D L D S S M Q S A D E S T P Q P L
K A N Q K P F F P K V F K K I K D A G K W V R D K
I D E N P E V K K A I V D K S A G L I D Q L L T K
K K S E E V N A S D F P P P P T D E E L R L A L P
E T P M L L G F N A P T P S E P S S F E F P P P P
T D E E L R L A L P E T P M L L G F N A P A T S E
P S S.

In another embodiment, a truncated ActA as set forth in SEQ ID NO: 13 is referred to as ActA/PEST3. In another embodiment, this truncated ActA comprises from the first 30 to amino acid 332 of the full length ActA sequence. In another embodiment, SEQ ID NO: 13 comprises from the first 30 to amino acid 332 of the full length ActA sequence. In another embodiment, a truncated ActA comprises from the first 30 to amino acid 332 of SEQ ID NO: 8. In another embodiment, SEQ ID NO: 13 comprises from the first 30 to amino acid 332 of SEQ ID NO: 8.

In another embodiment, a truncated ActA protein comprises the sequence set forth in SEQ ID NO: 14

(SEQ ID NO: 14)
A T D S E D S S L N T D E W E E E K T E E Q P S E
V N T G P R Y E T A R E V S S R D I E E L E K S N
K V K N T N K A D L I A M L K A K A E K G P N N N
N N N G E Q T G N V A I N E E A S G V D R P T L Q
V E R R H P G L S S D S A A E I K K R R K A I A S
S D S E L E S L T Y P D K P T K A N K R K V A K E
S V V D A S E S D L D S S M Q S A D E S T P Q P L
K A N Q K P K F P K V F K K I K D A G K W V R D K
I D E N P E V K K A I V D K S A G L I D Q L L T K
K K S E E V N A S D F P P P P T D E E L R L A L P
E T P M L L G F N A P T P S E P S S F E F P P P P
T D E E L R L A L P E T P M L L G F N A P A T S E
P S S F E F P P P P T E D E L E I M R E T A P S L
D S S F T S G D L A S L R S A I N R H S E N F S D
F P L I P T E E E L N G R G G R P T S E.

In another embodiment, a truncated ActA as set forth in SEQ ID NO: is referred to as ActA/PEST4. In another embodiment, this truncated ActA comprises from the first 30 to amino acid 399 of the full length ActA sequence. In another embodiment, SEQ ID NO: 14 comprises from the first 30 to amino acid 399 of the full length ActA sequence. In another embodiment, a truncated ActA comprises from the first 30 to amino acid 399 of SEQ ID NO: 8. In another embodiment, SEQ ID NO: 14 comprises from the first 30 to amino acid 399 of SEQ ID NO: 8.

In another embodiment, the recombinant nucleotide encoding a truncated ActA protein comprises the sequence set forth in SEQ ID NO: 15

(SEQ ID NO: 15)
atgcgtgcgatgatggtggttttcattactgccaattgcattacgattaa
ccccgacataatatttgcagcgacagatagcgaagattctagtctaaaca
cagatgaatgggaagaagaaaaaacagaagagcaaccaagcgaggtaaat
acgggaccaagatacgaaactgcacgtgaagtaagttcacgtgatattaa
agaactagaaaaatcgaataaagtgagaaatacgaacaaagcagacctaa
tagcaatgttgaaagaaaaagcagaaaaaggtccaaatatcaataataac
aacagtgaacaaactgagaatgcggctataaatgaagaggcttcaggagc
cgaccgaccagctatacaagtggagcgtcgtcatccaggattgccatcgg
atagcgcagcggaaattaaaaaaagaaggaaagccatagcatcatcggat
agtgagcttgaaagccttacttatccggataaaccaacaaaagtaaataa
gaaaaaagtggcgaaagagtcagttgcggatgcttctgaaagtgacttag
attctagcatgcagtcagcagatgagtcttcaccacaacctttaaaagca
aaccaacaaccatttttccctaaagtatttaaaaaaataaaagatgcggg
gaaatgggtacgtgataaaatcgacgaaaatcctgaagtaaagaaagcga
ttgttgataaaagtgcagggttaattgaccaattattaaccaaaaagaaa
agtgaagaggtaaatgcttcggacttcccgccaccacctacggatgaaga
gttaagacttgctttgccagagacaccaatgcttcttggttttaatgctc
ctgctacatcagaaccgagctcattcgaatttccaccaccacctacggat
gaagagttaagacttgctttgccagagacgccaatgcttcttggttttaa
tgctcctgctacatcggaaccgagctcgttcgaatttccaccgcctccaa
cagaagatgaactagaaatcatccgggaaacagcatcctcgctagattct
agttttacaagaggggatttagctagtttgagaaatgctattaatcgcca
tagtcaaaatttctctgatttcccaccaatcccaacagaagaagagttga
acgggagaggcggtagacca.

In another embodiment, the recombinant nucleotide has the sequence set forth in SEQ ID NO: 15. In another embodiment, the recombinant nucleotide comprises other sequences that encode a fragment of an ActA protein.

In one embodiment, “truncated ActA,” “N-terminal ActA fragment” or “AActA” are used interchangeably herein and refer to a fragment of ActA that comprises at least one PEST sequence. In another embodiment, the terms refer to an ActA fragment that comprises more than one PEST sequence. In another embodiment, the terms refer to a truncated ActA provided in SEQ ID NO: 9-14 herein.

The N-terminal ActA protein fragment of methods and compositions of the present invention comprises, in one embodiment, a sequence selected from SEQ ID No: 9-14. In another embodiment, the ActA fragment comprises an ActA signal peptide. In another embodiment, the ActA fragment consists approximately of a sequence selected from SEQ ID NO: 9-14. In another embodiment, the ActA fragment consists essentially of a sequence selected from SEQ ID NO: 9-14. In another embodiment, the ActA fragment corresponds to a sequence selected from SEQ ID NO: 9-14. In another embodiment, the ActA fragment is homologous to a sequence selected from SEQ ID NO: 9-14.

In another embodiment, a PEST sequence is any PEST AA sequence derived from a prokaryotic organism. The PEST sequence may be other PEST sequences known in the art.

In one embodiment, the ActA fragment consists of about residues 30-122 of the full length ActA protein sequence. In another embodiment, the ActA fragment consists of about residues 30-229 of the full length ActA protein sequence. In another embodiment, the ActA fragment consists of about residues 30-332 of the full length ActA protein sequence. In another embodiment, the ActA fragment consists of about residues 30-200. In another embodiment, the ActA fragment consists of about residues 30-399 of the full length ActA protein sequence.

In another embodiment, an ActA fragment provided herein contains residues of a homologous ActA protein that correspond to one of the above AA ranges. The residue numbers need not, in another embodiment, correspond exactly with the residue numbers enumerated above; e.g. if the homologous ActA protein has an insertion or deletion, relative to an ActA protein utilized herein, then the residue numbers can be adjusted accordingly.

In another embodiment, a homologous ActA refers to identity of an ActA sequence (e.g. to one of SEQ ID No: 12) of greater than 70%. In another embodiment, a homologous ActA refers to identity to one of SEQ ID No: 12 of greater than 72%. In another embodiment, a homologous refers to identity to one of SEQ ID No: 12 of greater than 75%. In another embodiment, a homologous refers to identity to one of SEQ ID No: 12 of greater than 78%. In another embodiment, a homologous refers to identity to one of SEQ ID No: 12 of greater than 80%. In another embodiment, a homologous refers to identity to one of SEQ ID No: 12 of greater than 82%. In another embodiment, a homologous refers to identity to one of SEQ ID No: 12 of greater than 83%. In another embodiment, a homologous refers to identity to one of SEQ ID No: 12 of greater than 85%. In another embodiment, a homologous refers to identity to one of SEQ ID No: 12 of greater than 87%. In another embodiment, a homologous refers to identity to one of SEQ ID No: 12 of greater than 88%. In another embodiment, a homologous refers to identity to one of SEQ ID No: 12 greater than 90%. In another embodiment, a homologous refers to identity to one of SEQ ID No: 12of greater than 92%. In another embodiment, a homologous refers to identity to one of SEQ ID No: 12 of greater than 93%. In another embodiment, a homologous refers to identity to one of SEQ ID No: 12 of greater than 95%. In another embodiment, a homologous refers to identity to one of SEQ ID No: 12 of greater than 96%. In another embodiment, a homologous refers to identity to one of SEQ ID No: 12 of greater than 97%. In another embodiment, a homologous refers to identity to one of SEQ ID No: 12 of greater than 98%. In another embodiment, a homologous refers to identity to one of SEQ ID No: 12of greater than 99%. In another embodiment, a homologous refers to identity to one of SEQ ID No: 12 of 100%.

It will be appreciated by a skilled artisan that the term “homology,” when in reference to any nucleic acid sequence provided herein may encompass a percentage of nucleotides in a candidate sequence that are identical with the nucleotides of a corresponding native nucleic acid sequence.

Homology may be determined by a computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from the sequences provided herein of greater than 68%. In another embodiment, “homology” refers to identity to a sequence selected from the sequences provided herein of greater than 70%. In another embodiment, “homology” refers to identity to a sequence selected from the sequences provided herein of greater than 72%. In another embodiment, the identity is greater than 75%. In another embodiment, the identity is greater than 78%. In another embodiment, the identity is greater than 80%. In another embodiment, the identity is greater than 82%. In another embodiment, the identity is greater than 83%. In another embodiment, the identity is greater than 85%. In another embodiment, the identity is greater than 87%. In another embodiment, the identity is greater than 88%. In another embodiment, the identity is greater than 90%. In another embodiment, the identity is greater than 92%. In another embodiment, the identity is greater than 93%. In another embodiment, the identity is greater than 95%. In another embodiment, the identity is greater than 96%. In another embodiment, the identity is greater than 97%. In another embodiment, the identity is greater than 98%. In another embodiment, the identity is greater than 99%. In another embodiment, the identity is 100%.

In another embodiment, an ActA protein, or a fragment thereof that are provided for in the present invention need not be that which is set forth exactly in the sequences set forth herein, but rather that other alterations, modifications, or changes can be made that retain the functional characteristics of an ActA protein fused to an antigen as set forth elsewhere herein. In another embodiment, the present invention utilizes an analog of an ActA protein, or fragment thereof. Analogs differ, in another embodiment, from naturally occurring proteins or peptides by conservative AA sequence differences or by modifications which do not affect sequence, or by both.

It will be appreciated by a skilled artisan that the term “Conservatively modified variants” or “conservative substitution” may encompass substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y). For example methods of hybridization may be carried out under moderate to stringent conditions, to the complement of a DNA encoding a native caspase peptide. Hybridization conditions being, for example, overnight incubation at 42 ° C. in a solution comprising: 10-20% formamide, 5×SSC (150 mM NaCl, 15 mM trisodium citrate), 50 mM sodium phosphate (pH 7. 6), 5× Denhardt's solution, 10% dextran sulfate, and 20 μg/mL denatured, sheared salmon sperm DNA.

In one embodiment, a recombinant Listeria strain provided herein lacks antibiotic resistance genes. In another embodiment, the recombinant Listeria strain provided herein comprises a plasmid comprising a nucleic acid encoding an antibiotic resistance gene. In another embodiment, the recombinant Listeria strain provided herein comprises a plasmid that does not encode an antibiotic resistance gene.

In one embodiment, a recombinant Listeria provided herein is capable of escaping the phagolysosome.

In one embodiment, a polypeptide provided herein is a fusion protein comprising an additional polypeptide selected from the group consisting of: a PEST sequence, or an ActA protein or a fragment thereof. In another embodiment, an additional polypeptide is fused to an antigen provided herein or known in the art. In another embodiment, an additional polypeptide is functional, which encompasses the polypeptide being immunogenic, as will be understood by a skilled artisan.

In one embodiment, a nucleic acid sequence encoding an antigen or fragment thereof is integrated in frame with a truncated ActA provide herein in the Listeria chromosome. In another embodiment, an integrated nucleic acid sequence encoding an antigen or fragment thereof is integrated in frame with ActA at the actA locus.

In one embodiment, a nucleic acid molecule provided herein comprises a first open reading frame encoding a recombinant polypeptide comprising a heterologous antigen or fragment thereof. In another embodiment, the recombinant polypeptide further comprises a truncated ActA protein or PEST sequence peptide fused to a heterologous antigen or an antigenic portion thereof.

In one embodiment, a nucleic acid molecule provided herein further comprises a second open reading frame encoding a metabolic enzyme. In another embodiment, the metabolic enzyme complements a mutation in the chromosome of the recombinant Listeria strain. In another embodiment, the metabolic enzyme encoded by the second open reading frame is an alanine racemase enzyme (dal). In another embodiment, the metabolic enzyme encoded by the second open reading frame is a D-amino acid transferase enzyme (dat). In another embodiment, the Listeria strains provided herein comprise a mutation, deletion or inactivation in the endogenous dal/dat genes. In another embodiment, the Listeria lacks the dal/dat genes. In another embodiment, the Listeria lacks the dal/dat and actA genes. In another embodiment, the Listeria comprises a mutation, deletion or inactivation in the dal/dat and actA genes.

In another embodiment, a nucleic acid molecule of the methods and compositions of the present invention is operably linked to a promoter/regulatory sequence. In another embodiment, the first open reading frame of the nucleic acid molecule provided herein is operably linked to a promoter/regulatory sequence. In another embodiment, the second open reading frame of the nucleic acid molecule provided herein is operably linked to a promoter/regulatory sequence. In another embodiment, the third open reading frame of the nucleic acid molecule provided herein is operably linked to a promoter/regulatory sequence. In another embodiment, each of the open reading frames of the nucleic acid molecule provided herein is operably linked to a promoter/regulatory sequence.

It will be appreciated by a skilled artisan that the term “operably linked” may encompass a transcriptional and translational regulatory nucleic acid that is positioned relative to any coding sequences in such a manner that transcription is initiated. Generally, this will mean that the promoter and transcriptional initiation or start sequences are positioned 5′ to the coding region. In another embodiment, the term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

It will be appreciated by a skilled artisan that the term “metabolic enzyme” may encompass an enzyme involved in synthesis of a nutrient required by the host bacteria. In one embodiment, the term refers to an enzyme required for synthesis of a nutrient required by the host bacteria. In another embodiment, the term refers to an enzyme involved in synthesis of a nutrient utilized by the host bacteria. In another embodiment, the term refers to an enzyme involved in synthesis of a nutrient required for sustained growth of the host bacteria. In another embodiment, the enzyme is required for synthesis of the nutrient.

In another embodiment, a recombinant Listeria is an attenuated auxotrophic strain. In another embodiment, a recombinant auxotrophic strain comprises strains described in U.S. Pat. No. 8,114,414, which is incorporated by reference herein in its entirety. In one embodiment, the attenuated strain is Lm dal(-)dat(-) (Lmdd). In another embodiment, the attenuated strains is Lm dal(-)dat(-) ΔactA (LmddA). LmddA is based on a Listeria vaccine vector which is attenuated due to the deletion of virulence gene actA and retains the plasmid in vivo and in vitro by complementation of dal gene.

In one embodiment, an attenuated auxotrophic Listeria vaccine strain is based on a Listeria vaccine vector which is attenuated due to the deletion of virulence gene actA and retains the plasmid for antigen expression in vivo and in vitro by complementation of dal gene. In another embodiment, the Listeria is a dal/dat/actA Listeria having a mutation in the dal, dat and actA endogenous genes. In another embodiment, the mutation is a deletion, a truncation or an inactivation of the mutated genes.

In another embodiment, the dal/dat/actA mutant Listeria strain is highly attenuated and has a better safety profile than previous Listeria vaccine generation, as it is more rapidly cleared from the spleens of the immunized mice. In another embodiment, the dal/dat/actA mutant Listeria strain results in a longer delay of tumor onset in transgenic animals than Listeria vaccines based on more virulent antibiotic resistant strains (see US Publication No. 2011/0142791, which is incorporated by reference herein in its entirety). In another embodiment, the dal/dat/actA mutant Listeria strain causes a significant decrease in intra-tumoral T regulatory cells (Tregs). In another embodiment, the lower frequency of Tregs in tumors treated with LmddA vaccines result in an increased intratumoral CD8⁺/Tregs ratio, suggesting that a more favorable tumor microenvironment can be obtained after immunization with LmddA vaccines.

In another embodiment, the terms “LmddA”, “LmΔddA” or “dal/dat/actA mutant Listeria” are used interchangeably herein.

In another embodiment, the attenuated strain is LmΔactA. In another embodiment, the attenuated strain is LmΔprfA. In another embodiment, the attenuated strain is LmΔplcB. In another embodiment, the attenuated strain is LmΔplcA. In another embodiment, the attenuated strain is LmΔinlA. In another embodiment, the attenuated strain is LmAinlB. In another embodiment, the attenuated strain is LmΔinlC. In another embodiment, the strain is the double mutant or triple mutant of any of the above-mentioned strains. In another embodiment, this strain exerts a strong adjuvant effect which is a property of Listeria-based vaccines. In another embodiment, this strain is constructed from the EGD Listeria backbone. In another embodiment, the strain used in the invention is a Listeria strain that expresses a truncated ActA protein provided herein or a fragment thereof.

In another embodiment, a Listeria strain provided herein is an auxotrophic mutant. In another embodiment, the Listeria strain is deficient in a gene encoding a vitamin synthesis gene. In another embodiment, the Listeria strain is deficient in a gene encoding pantothenic acid synthase.

In one embodiment, a Listeria strain provided herein is deficient in an amino acid (AA) metabolism enzyme. In another embodiment, the generation of auxotrophic strains of Listeria deficient in D-alanine, for example, may be accomplished in a number of ways that are well known to those of skill in the art, including deletion mutagenesis, insertion mutagenesis, and mutagenesis which results in the generation of frameshift mutations, mutations which cause premature termination of a protein, or mutation of regulatory sequences which affect gene expression. In another embodiment, mutagenesis can be accomplished using recombinant DNA techniques or using traditional mutagenesis technology using mutagenic chemicals or radiation and subsequent selection of mutants. In another embodiment, deletion mutants are preferred because of the accompanying low probability of reversion of the auxotrophic phenotype. In another embodiment, mutants of D-alanine which are generated according to the protocols presented herein may be tested for the ability to grow in the absence of D-alanine in a simple laboratory culture assay. In another embodiment, those mutants which are unable to grow in the absence of this compound are selected for further study.

In another embodiment, in addition to the aforementioned D-alanine associated genes, other genes involved in synthesis of a metabolic enzyme, as provided herein, may be used as targets for mutagenesis of Listeria.

In one embodiment, a metabolic enzyme complements a mutation, deletion or inactivation of a gene encoding a metabolic enzyme in the chromosome of the recombinant Listeria strain. In another embodiment, a metabolic enzyme is an amino acid metabolism enzyme. In another embodiment, a metabolic enzyme catalyzes a formation of an amino acid used for a cell wall synthesis in the recombinant Listeria strain. In another embodiment, a metabolic enzyme is an alanine racemase enzyme. In another embodiment, a metabolic enzyme is a D-amino acid transferase enzyme.

It will be appreciated by a skilled artisan that the term “episomal expression vector” or “extrachromosomal plasmid” or “plasmid” are used interchangeably herein and may encompass a nucleic acid vector which may be linear or circular, and which is usually double-stranded in form. In one embodiment, an episomal expression vector comprises a gene of interest. In another embodiment, the inserted gene of interest is not interrupted or subjected to regulatory constraints which often occur from integration into cellular DNA. In another embodiment, the presence of the inserted heterologous gene does not lead to rearrangement or interruption of the cell's own important regions. In another embodiment, episomal vectors persist in multiple copies in the bacterial cytoplasm, resulting in amplification of the gene of interest, and, in another embodiment, viral trans-acting factors are supplied when necessary. In another embodiment, in stable transfection procedures, the use of episomal vectors often results in higher transfection efficiency than the use of chromosome-integrating plasmids (Belt, P. B. G. M., et al (1991) Efficient cDNA cloning by direct phenotypic correction of a mutant human cell line (HPRT2) using an Epstein-Barr virus-derived cDNA expression vector. Nucleic Acids Res. 19, 4861-4866; Mazda, O., et al. (1997) Extremely efficient gene transfection into lympho-hematopoietic cell lines by Epstein-Barr virus-based vectors. J. Immunol. Methods 204, 143-151). In one embodiment, the episomal expression vectors of the methods and compositions as provided herein may be delivered to cells in vivo, ex vivo, or in vitro by any of a variety of the methods employed to deliver DNA molecules to cells. The vectors may also be delivered alone or in the form of a pharmaceutical composition that enhances delivery to cells of a subject.

In one embodiment, an auxotrophic Listeria strain provided herein comprises an episomal expression vector comprising a metabolic enzyme that complements the auxotrophy of the auxotrophic Listeria strain. In another embodiment, the construct is contained in the Listeria strain in an episomal or extrachromosomal fashion. In another embodiment, the foreign antigen is expressed from a vector harbored by the recombinant Listeria strain. In another embodiment, the episomal expression vector lacks an antibiotic resistance marker. In one embodiment, an antigen is fused to a polypeptide comprising a PEST sequence. In another embodiment, the polypeptide comprising a PEST sequence is a truncated ActA protein.

In another embodiment, a Listeria strain provided herein is deficient in an AA metabolism enzyme. In another embodiment, the Listeria strain is deficient in a D-glutamic acid synthase gene. In another embodiment, the Listeria strain is deficient in a D-alanine amino transferase (dat) gene. In another embodiment, the Listeria strain is deficient in a D-alanine racemase (dal) gene. In another embodiment, the Listeria strain is deficient in the dga gene. In another embodiment, the Listeria strain is deficient in a gene involved in the synthesis of diaminopimelic acid (DAP). In another embodiment, the Listeria strain is deficient in a gene involved in the synthesis of Cysteine synthase

A (CysK). In another embodiment, the gene is vitamin-B12 independent methionine synthase. In another embodiment, the gene is trpA. In another embodiment, the gene is trpB. In another embodiment, the gene is trpE. In another embodiment, the gene is asnB. In another embodiment, the gene is gltD. In another embodiment, the gene is gltB. In another embodiment, the gene is leuA. In another embodiment, the gene is argG. In another embodiment, the gene is thrC. In another embodiment, the Listeria strain is deficient in one or more of the genes described hereinabove.

In another embodiment, a Listeria strain provided herein is deficient in a synthase gene. In another embodiment, the gene is an AA synthesis gene. In another embodiment, the gene is folP. In another embodiment, the gene is dihydrouridine synthase family protein. In another embodiment, the gene is ispD. In another embodiment, the gene is ispF. In another embodiment, the gene is phosphoenolpyruvate synthase. In another embodiment, the gene is hisF. In another embodiment, the gene is hisH. In another embodiment, the gene is fliI. In another embodiment, the gene is ribosomal large subunit pseudouridine synthase. In another embodiment, the gene is ispD. In another embodiment, the gene is bifunctional GMP synthase/glutamine amidotransferase protein. In another embodiment, the gene is cobS. In another embodiment, the gene is cobB. In another embodiment, the gene is cbiD. In another embodiment, the gene is uroporphyrin-III C-methyltransferase/uroporphyrinogen-III synthase. In another embodiment, the gene is cobQ. In another embodiment, the gene is uppS. In another embodiment, the gene is truB. In another embodiment, the gene is dxs. In another embodiment, the gene is mvaS. In another embodiment, the gene is dapA. In another embodiment, the gene is ispG. In another embodiment, the gene is folC. In another embodiment, the gene is citrate synthase. In another embodiment, the gene is argJ. In another embodiment, the gene is 3-deoxy-7-phosphoheptulonate synthase. In another embodiment, the gene is indole-3-glycerol-phosphate synthase. In another embodiment, the gene is anthranilate synthase/glutamine amidotransferase component. In another embodiment, the gene is menB. In another embodiment, the gene is menaquinone-specific isochorismate synthase. In another embodiment, the gene is phosphoribosylformylglycinamidine synthase I or II. In another embodiment, the gene is phosphoribosylaminoimidazole-succinocarboxamide synthase. In another embodiment, the gene is carB. In another embodiment, the gene is carA. In another embodiment, the gene is thyA. In another embodiment, the gene is mgsA. In another embodiment, the gene is aroB. In another embodiment, the gene is hepB. In another embodiment, the gene is rluB. In another embodiment, the gene is ilvB. In another embodiment, the gene is ilvN. In another embodiment, the gene is alsS. In another embodiment, the gene is fabF. In another embodiment, the gene is fabH. In another embodiment, the gene is pseudouridine synthase. In another embodiment, the gene is pyrG. In another embodiment, the gene is truA. In another embodiment, the gene is pabB. In another embodiment, the gene is an atp synthase gene (e.g. atpC, atpD-2, aptG, atpA-2, etc).

In another embodiment, the gene is phoP. In another embodiment, the gene is aroA. In another embodiment, the gene is aroC. In another embodiment, the gene is aroD. In another embodiment, the gene is plcB.

In another embodiment, a Listeria strain provided herein is deficient in a peptide transporter. In another embodiment, the gene is ABC transporter/ATP-binding/permease protein. In another embodiment, the gene is oligopeptide ABC transporter/oligopeptide-binding protein. In another embodiment, the gene is oligopeptide ABC transporter/permease protein. In another embodiment, the gene is zinc ABC transporter/zinc-binding protein. In another embodiment, the gene is sugar ABC transporter. In another embodiment, the gene is phosphate transporter. In another embodiment, the gene is ZIP zinc transporter. In another embodiment, the gene is drug resistance transporter of the EmrB/QacA family. In another embodiment, the gene is sulfate transporter. In another embodiment, the gene is proton-dependent oligopeptide transporter. In another embodiment, the gene is magnesium transporter. In another embodiment, the gene is formate/nitrite transporter. In another embodiment, the gene is spermidine/putrescine ABC transporter. In another embodiment, the gene is Na/Pi-cotransporter. In another embodiment, the gene is sugar phosphate transporter. In another embodiment, the gene is glutamine ABC transporter. In another embodiment, the gene is major facilitator family transporter. In another embodiment, the gene is glycine betaine/L-proline ABC transporter. In another embodiment, the gene is molybdenum ABC transporter. In another embodiment, the gene is techoic acid ABC transporter. In another embodiment, the gene is cobalt ABC transporter. In another embodiment, the gene is ammonium transporter. In another embodiment, the gene is amino acid ABC transporter. In another embodiment, the gene is cell division ABC transporter. In another embodiment, the gene is manganese ABC transporter. In another embodiment, the gene is iron compound ABC transporter. In another embodiment, the gene is maltose/maltodextrin ABC transporter. In another embodiment, the gene is drug resistance transporter of the Bcr/CflA family. In another embodiment, the gene is a subunit of one of the above proteins.

In one embodiment, provided herein is a nucleic acid molecule that is used to transform the Listeria in order to arrive at a recombinant Listeria. In another embodiment, the nucleic acid provided herein used to transform Listeria lacks a virulence gene. In another embodiment, the nucleic acid molecule is integrated into the Listeria genome and carries a non-functional virulence gene. In another embodiment, the virulence gene is mutated in the recombinant Listeria. In yet another embodiment, the nucleic acid molecule is used to inactivate the endogenous gene present in the Listeria genome. In yet another embodiment, the virulence gene is an actA gene, an inlA gene, and in1B gene, an in1C gene, inlJ gene, a plbC gene, a bsh gene, or a prfA gene. It is to be understood by a skilled artisan, that the virulence gene can be any gene known in the art to be associated with virulence in the recombinant Listeria.

In one embodiment, a live attenuated Listeria provided herein is a recombinant Listeria. In another embodiment, a recombinant Listeria provided herein comprises a mutation of a genomic internalin C (inlC) gene. In another embodiment, the recombinant Listeria comprises a mutation or a deletion of a genomic actA gene and a genomic internalin C gene. In one embodiment, translocation of Listeria to adjacent cells is inhibited by the deletion of the actA gene and/or the inlC gene, which are involved in the process, thereby resulting in unexpectedly high levels of attenuation with increased immunogenicity and utility as a strain backbone.

In one embodiment, a live attenuated Listeria provided herein is a recombinant Listeria. In another embodiment, a recombinant Listeria provided herein comprises a mutation of a genomic internalin B (in1B) gene. In another embodiment, the recombinant Listeria comprises a mutation or a deletion of a genomic actA gene and a genomic internalin B gene. In one embodiment, translocation of Listeria to adjacent cells is inhibited by the deletion of the actA gene and/or the in1B gene, which are involved in the process, thereby resulting in unexpectedly high levels of attenuation with increased immunogenicity and utility as a strain backbone.

It will be appreciated by a skilled artisan that the term “attenuation,” may encompass a diminution in the ability of the bacterium to cause disease in an animal. In other words, for example the pathogenic characteristics of the attenuated Listeria strain have been lessened compared with wild-type Listeria, although the attenuated Listeria is capable of growth and maintenance in culture. Using as an example the intravenous inoculation of Balb/c mice with an attenuated Listeria, the lethal dose at which 50% of inoculated animals survive (LD₅₀) is preferably increased above the LD50 of wild-type Listeria by at least about 10-fold, more preferably by at least about 100-fold, more preferably at least about 1,000 fold, even more preferably at least about 10,000 fold, and most preferably at least about 100,000-fold. An attenuated strain of Listeria is thus one which does not kill an animal to which it is administered, or is one which kills the animal only when the number of bacteria administered is vastly greater than the number of wild type non-attenuated bacteria which would be required to kill the same animal. An attenuated bacterium should also be construed to mean one which is incapable of replication in the general environment because the nutrient required for its growth is not present therein. Thus, the bacterium is limited to replication in a controlled environment wherein the required nutrient is provided. The attenuated strains of the present invention are therefore environmentally safe in that they are incapable of uncontrolled replication.

In yet another embodiment, a Listeria strain provided herein is an inlA mutant, an in/B mutant, an inlC mutant, an inlJ mutant, prfA mutant, actA mutant, a dal/dat mutant, a prfA mutant, a plcB deletion mutant, or a double mutant lacking both plcA and plcB. In another embodiment, the Listeria comprises a deletion or mutation of these genes individually or in combination. In another embodiment, the Listeria provided herein lack each one of genes. In another embodiment, the Listeria provided herein lack at least one and up to ten of any gene provided herein, including the actA, prfA, and dal/dat genes. In another embodiment, a prfA mutant is a D133V prfA mutant as described in PCT/US15/25690, which is hereby incorporated by reference herein.

In one embodiment, a metabolic gene, or a virulence gene is lacking in a chromosome of the Listeria strain. In another embodiment, the metabolic gene, or virulence gene is lacking in the genome of the virulence strain. In one embodiment, the virulence gene is mutated in the chromosome. In another embodiment, the virulence gene is deleted from the chromosome. In another embodiment, the metabolic gene, or the virulence gene is mutated in a chromosome of the Listeria strain. In another embodiment, the metabolic gene, or the virulence gene is mutated in the genome of the virulence strain.

In one embodiment, a recombinant Listeria strain provided herein is attenuated. In another embodiment, the recombinant Listeria lacks the actA virulence gene. In another embodiment, the recombinant Listeria lacks the prfA virulence gene. In another embodiment, the recombinant Listeria lacks the in1B gene. In another embodiment, the recombinant Listeria lacks both, the actA and in1B genes. In another embodiment, the recombinant Listeria strain provided herein comprises an inactivating mutation of the endogenous actA gene. In another embodiment, the recombinant Listeria strain provided herein comprises an inactivating mutation of the endogenous in1B gene. In another embodiment, the recombinant Listeria strain provided herein comprises an inactivating mutation of the endogenous in/C gene. In another embodiment, the recombinant Listeria strain provided herein comprises an inactivating mutation of the endogenous actA and in1B genes. In another embodiment, the recombinant Listeria strain provided herein comprises an inactivating mutation of the endogenous actA and in/C genes. In another embodiment, the recombinant Listeria strain provided herein comprises an inactivating mutation of the endogenous actA, in/B, or in1C genes. In another embodiment, the recombinant Listeria strain provided herein comprises an inactivating mutation of the endogenous actA, inlB, and inlC genes. In another embodiment, the recombinant Listeria strain provided herein comprises an deletion of the endogenous actA, inlB, and inlC genes. In another embodiment, the recombinant Listeria strain provided herein comprises an inactivating or loss of function mutation or a deletion in any single gene or combination of the following genes: actA, dal, dat, inlB, inlC, prfA, plcA, plcB.

It will be appreciated by a skilled artisan that the term “mutation” and grammatical equivalents thereof, include any type of mutation or modification to the sequence (nucleic acid or amino acid sequence), and may encompass a deletion mutation, a truncation, an inactivation or loss of function, a disruption, or a translocation. These types of mutations are readily known in the art.

In one embodiment, in order to select for auxotrophic bacteria comprising a plasmid encoding a metabolic enzyme or a complementing gene provided herein, transformed auxotrophic bacteria are grown on a media that will select for expression of the amino acid metabolism gene or the complementing gene. In another embodiment, a bacteria auxotrophic for D-glutamic acid synthesis is transformed with a plasmid comprising a gene for D-glutamic acid synthesis, and the auxotrophic bacteria will grow in the absence of D-glutamic acid, whereas auxotrophic bacteria that have not been transformed with the plasmid, or are not expressing the plasmid encoding a protein for D-glutamic acid synthesis, will not grow. In another embodiment, a bacterium auxotrophic for D-alanine synthesis will grow in the absence of D-alanine when transformed and expressing the plasmid of the present invention if the plasmid comprises an isolated nucleic acid encoding an amino acid metabolism enzyme for D-alanine synthesis. Such methods for making appropriate media comprising or lacking necessary growth factors, supplements, amino acids, vitamins, antibiotics, and the like are well known in the art, and are available commercially (Becton-Dickinson, Franklin Lakes, N.J.).

In another embodiment, once the auxotrophic bacteria comprising the plasmid of the present invention have been selected on appropriate media, the bacteria are propagated in the presence of a selective pressure. Such propagation comprises growing the bacteria in media without the auxotrophic factor. The presence of the plasmid expressing an amino acid metabolism enzyme in the auxotrophic bacteria ensures that the plasmid will replicate along with the bacteria, thus continually selecting for bacteria harboring the plasmid. The skilled artisan, when equipped with the present disclosure and methods herein will be readily able to scale-up the production of the Listeria vaccine vector by adjusting the volume of the media in which the auxotrophic bacteria comprising the plasmid are growing.

The skilled artisan will appreciate that, in another embodiment, other auxotroph strains and complementation systems are adopted for the use with the methods and compositions provided herein.

In one embodiment, a recombinant Listeria strain provided herein expresses a recombinant polypeptide. In another embodiment, a recombinant Listeria strain comprises a plasmid that encodes a recombinant polypeptide. In another embodiment, a recombinant nucleic acid provided herein is in a plasmid in the recombinant Listeria strain provided herein. In another embodiment, the plasmid is an episomal plasmid that does not integrate into the recombinant Listeria strain's chromosome. In another embodiment, the plasmid is an integrative plasmid that integrates into the Listeria strain's chromosome. It will be understood by a skilled artisan that a plasmid provided herein may be a multicopy plasmid.

In one embodiment, nucleic acids encoding the recombinant polypeptides provided herein also encode a signal peptide or sequence. In another embodiment, the fusion protein of methods and compositions of the present invention comprises an LLO signal sequence. In another embodiment, the fusion protein of methods and compositions of the present invention comprises an actA signal sequence. In one embodiment, a heterologous antigen may be expressed in Listeria through the use of a signal sequence, such as a Listerial signal sequence, for example, the hemolysin signal sequence or the ActA signal sequence. Alternatively, for example, foreign genes can be expressed downstream from a L. monocytogenes promoter without creating a fusion protein. In another embodiment, the signal peptide is bacterial (Listerial or non-Listerial). In one embodiment, the signal peptide is native to the bacterium. In another embodiment, the signal peptide is foreign to the bacterium. In another embodiment, the signal peptide is a signal peptide from Listeria monocytogenes, such as a secA1 signal peptide. In another embodiment, the signal peptide is an Usp45 signal peptide from Lactococcus lactis, or a Protective Antigen signal peptide from Bacillus anthracis. In another embodiment, the signal peptide is a secA2 signal peptide, such the p60 signal peptide from Listeria monocytogenes. In addition, the recombinant nucleic acid molecule optionally comprises a third polynucleotide sequence encoding p60, or a fragment thereof. In another embodiment, the signal peptide is a Tat signal peptide, such as a B. subtilis Tat signal peptide (e.g., PhoD). In one embodiment, the signal peptide is in the same translational reading frame encoding the recombinant polypeptide.

It will be appreciated by a skilled artisan that the term “homologue” may encompass a nucleic acid or amino acid sequence which shares a certain percentage of sequence identity with a particular nucleic acid or amino acid sequence. In one embodiment, a sequence useful in the composition and methods as provided herein may be a homologue of a particular ActA sequence or N-terminal fragment thereof. In another embodiment, a sequence useful in the composition and methods as provided herein may be a homologue of an antigenic polypeptide or an immunogenic fragment thereof. In one embodiment, a homolog of a polypeptide and, in one embodiment, the nucleic acid encoding such a homolog, of the present invention maintains the functional characteristics of the parent polypeptide. For example, in one embodiment, a homolog of an antigenic polypeptide provided herein maintains the antigenic characteristic of the parent polypeptide. In another embodiment, a sequence useful in the composition and methods as provided herein may be a homologue of any sequence described herein. In one embodiment, a homologue shares at least 70%-85% identity with a particular sequence. In another embodiment, a homologue shares at least 85%-95% identity with a particular sequence. In another embodiment, a homologue shares at least 96% identity with a particular sequence. In another embodiment, a homologue shares at least 97% identity with a particular sequence. In another embodiment, a homologue shares at least 98% identity with a particular sequence. In another embodiment, a homologue shares at least 99% identity with a particular sequence. In another embodiment, a homologue shares 100% identity with a particular sequence.

In one embodiment, it is to be understood that a homolog of any of the sequences provided herein and/or described herein are considered to be encompassed by the present invention.

In another embodiment, a recombinant Listeria strain of the methods and compositions provided herein comprise a nucleic acid molecule operably integrated into the Listeria genome as an open reading frame with an endogenous ActA sequence. In another embodiment, a recombinant Listeria strain of the methods and compositions provided herein comprise an episomal expression vector comprising a nucleic acid molecule encoding fusion protein comprising an antigen fused to an ActA or a truncated ActA. In one embodiment, the expression and secretion of the antigen is under the control of an ActA promoter and ActA signal sequence and it is expressed as fusion to a sequence selected from SEQ ID NO: 9-14 provided herein. In another embodiment, the expression and secretion of the antigen is under the control of an ActA promoter and ActA signal sequence and it is expressed as fusion to a sequence selected from SEQ ID NO: 9-14 provided herein. In one embodiment, the expression and secretion of the antigen is under the control of an ActA promoter and ActA signal sequence and it is expressed as fusion to a sequence of about amino acid 30 to amino acid 229 of the full length ActA sequence (see SEQ ID NO: 12). In one embodiment, the expression and secretion of the antigen is under the control of an My promoter and hly signal sequence and it is expressed as fusion to a sequence selected from SEQ ID NO: 9-14 provided herein. In another embodiment, the expression and secretion of the antigen is under the control of an hly promoter and hly signal sequence and it is expressed as fusion to a sequence selected from SEQ ID NO: 9-14 provided herein. In one embodiment, the expression and secretion of the antigen is under the control of an hly promoter and hly signal sequence and it is expressed as fusion to a sequence of about amino acid 30 to amino acid 229 of the full length ActA sequence (see SEQ ID NO: 13). In another embodiment, the truncated ActA consists of the first 390 amino acids of the wild type ActA protein as described in U.S. Pat. Ser. No. 7,655,238, which is incorporated by reference herein in its entirety. In another embodiment, the truncated ActA is an ActA-N100 or a modified version thereof (referred to as ActA-N100*) in which a PEST motif has been deleted and containing the nonconservative QDNKR substitution as described in US Patent Publication Serial No. 2014/0186387.

In one embodiment, the present invention provides a recombinant polypeptide comprising an N-terminal fragment of an ActA protein fused to an antigen or to a fragment thereof. In another embodiment, the present invention provides a recombinant polypeptide consisting of an N-terminal fragment of an ActA protein fused to an antigen or fused to a fragment thereof. In another embodiment, the present invention provides a recombinant polypeptide consisting of an N-terminal fragment of an ActA protein selected from SEQ ID NOs: 9-14 fused to an antigen or fused to a fragment thereof.

In one embodiment, the present invention provides a recombinant polypeptide comprising an antigen or a fragment thereof fused to a PEST amino acid sequence. In another embodiment, a recombinant polypeptide comprises an antigen or a fragment thereof fused to 1-2 PEST amino acid sequences. In another embodiment, a recombinant polypeptide comprises an antigen or a fragment thereof fused to 2-3 PEST amino acid sequences. In another embodiment, a recombinant polypeptide comprises an antigen or a fragment thereof fused to 3-4 PEST amino acid sequences.

Protein and/or peptide homology for any amino acid sequence listed herein is determined, in one embodiment, by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example.

In another embodiment, a construct or nucleic acid molecule provided herein is integrated into the Listerial chromosome using homologous recombination. Techniques for homologous recombination are well known in the art, and are described, for example, in Baloglu S, Boyle S M, et al. (Immune responses of mice to vaccinia virus recombinants expressing either Listeria monocytogenes partial listeriolysin or Brucella abortus ribosomal L7/L12 protein. Vet Microbiol 2005, 109(1-2): 11-7); and Jiang L L, Song H H, et al., (Characterization of a mutant Listeria monocytogenes strain expressing green fluorescent protein. Acta Biochim Biophys Sin (Shanghai) 2005, 37(1): 19-24). In another embodiment, homologous recombination is performed as described in U.S. Pat. No. 6,855,320. In another embodiment, a temperature sensitive plasmid is used to select the recombinants. Each technique represents a separate embodiment of the present invention.

It will be appreciated by a skilled artisan that the temm “recombination site” or “site-specific recombination site” may encompass a sequence of bases in a nucleic acid molecule that is recognized by a recombinase (along with associated proteins, in some cases) that mediates exchange or excision of the nucleic acid segments flanking the recombination sites. The recombinases and associated proteins are collectively referred to as “recombination proteins” see, e.g., Landy, A., (Current Opinion in Genetics & Development) 3:699-707; 1993).

In another embodiment, a construct or nucleic acid molecule provided herein is integrated into the Listerial chromosome using transposon insertion. Techniques for transposon insertion are well known in the art, and are described, inter alia, by Sun et al. (Infection and Immunity 1990, 58: 3770-3778) in the construction of DP-L967. Transposon mutagenesis has the advantage, in another embodiment, that a stable genomic insertion mutant can be formed but the disadvantage that the position in the genome where the foreign gene has been inserted is unknown.

In another embodiment, the construct or nucleic acid molecule is integrated into the Listerial chromosome using phage integration sites (Lauer P, Chow M Y et al, Construction, characterization, and use of two Listeria monocytogenes site-specific phage integration vectors. J Bacteriol 2002; 184(15): 4177-86). In certain embodiments of this method, an integrase gene and attachment site of a bacteriophage (e.g. U153 or PSA listeriophage) is used to insert the heterologous gene into the corresponding attachment site, which may be any appropriate site in the genome (e.g. comK or the 3′ end of the arg tRNA gene). In another embodiment, endogenous prophages are cured from the attachment site utilized prior to integration of the construct or heterologous gene. In another embodiment, this method results in single-copy integrants. In another embodiment, the present invention further comprises a phage based chromosomal integration system for clinical applications, where a host strain that is auxotrophic for essential enzymes, including, but not limited to, D-alanine racemase can be used, for example Lm dal(-)dat(-). In another embodiment, in order to avoid a “phage curing step,” a phage integration system based on PSA is used (Lauer, et al., 2002 J Bacteriol, 184:4177-4186). This requires, in another embodiment, continuous selection by antibiotics to maintain the integrated gene. Thus, in another embodiment, the current invention enables the establishment of a phage based chromosomal integration system that does not require selection with antibiotics. Instead, an auxotrophic host strain can be complemented.

It will be appreciated by a skilled artisan that the term “phage expression vector” “ may encompass any phage-based recombinant expression system for the purpose of expressing a nucleic acid sequence of the methods and compositions as provided herein in vitro or in vivo, constitutively or inducibly, in any cell, including prokaryotic, yeast, fungal, plant, insect or mammalian cell. A phage expression vector typically can both reproduce in a bacterial cell and, under proper conditions, produce phage particles. The term includes linear or circular expression systems and encompasses both phage-based expression vectors that remain episomal or integrate into the host cell genome.

In another embodiment, conjugation is used to introduce genetic material and/or plasmids into bacteria. Methods for conjugation are well known in the art, and are described, for example, in Nikodinovic J et al (A second generation snp-derived Escherichia coli-Streptomyces shuttle expression vector that is generally transferable by conjugation. Plasmid. 2006 November; 56(3):223-7) and Auchtung J M et al (Regulation of a Bacillus subtilis mobile genetic element by intercellular signaling and the global DNA damage response. Proc Natl Acad Sci U S A. 2005 Aug 30;102(35):12554-9). Each method represents a separate embodiment of the methods and compositions as provided herein.

It will be understood by a skilled artisan that antibiotic resistance genes may be used in the conventional selection and cloning processes commonly employed in molecular biology and vaccine preparation. Antibiotic resistance genes contemplated in the present invention include, but are not limited to, gene products that confer resistance to ampicillin, penicillin, methicillin, streptomycin, erythromycin, kanamycin, tetracycline, chloramphenicol (CAT), neomycin, hygromycin, gentamicin and others well known in the art.

Plasmids and other expression vectors useful in the present invention are described elsewhere herein, and can include such features as a promoter/regulatory sequence, an origin of replication for gram negative and gram positive bacteria, a ribosome binding site and a transcription termination signal, as well as a recombinant nucleic acid or open reading frame encoding a fusion protein and a nucleic acid or open reading frame encoding an amino acid metabolism gene. Further, an nucleic acid encoding a fusion protein and an amino acid metabolism gene will have a promoter suitable for driving expression of such a nucleic acid. Promoters useful for driving expression in a bacterial system are well known in the art, and include bacteriophage lambda, the bla promoter of the beta-lactamase gene of pBR322, and the CAT promoter of the chloramphenicol acetyl transferase gene of pBR325. Further examples of prokaryotic promoters include the major right and left promoters of 5 bacteriophage lambda (PL and PR), the trp, recA, lacZ, lad, and gal promoters of E. coli, the alpha-amylase (Ulmanen et al, 1985. J. Bacteriol. 162:176-182) and the S28-specific promoters of B. subtilis (Gilman et al, 1984 Gene 32:11-20), the promoters of the bacteriophages of Bacillus (Gryczan, 1982, In: The Molecular Biology of the Bacilli, Academic Press, Inc., New York), and Streptomyces promoters (Ward et al, 1986, Mol. Gen. Genet. 203:468-478). Additional prokaryotic promoters contemplated in the present invention are reviewed in, for example, Glick (1987, J. Ind. Microbiol. 1:277-282); Cenatiempo, (1986, Biochimie, 68:505-516); and Gottesman, (1984, Ann. Rev. Genet. 18:415-442). Further examples of promoter/regulatory elements contemplated in the present invention include, but are not limited to the Listerial prfA promoter, the Listerial hly promoter, the Listerial p60 promoter and the Listerial actA promoter (GenBank Acc. No. NC_003210) or fragments thereof.

In another embodiment, a plasmid of methods and compositions of the present invention comprises a gene encoding a fusion protein. In another embodiment, subsequences are cloned and the appropriate subsequences cleaved using appropriate restriction enzymes. The fragments are then, in another embodiment, ligated to produce the desired DNA sequence. In another embodiment, DNA encoding the antigen is produced using DNA amplification methods, for example polymerase chain reaction (PCR), as discussed below

In another embodiment, DNA encoding the fusion protein or the recombinant protein of the present invention is cloned using DNA amplification methods such as polymerase chain reaction (PCR). Thus, for example, the gene for a truncated ActA is PCR amplified, using a sense primer comprising a suitable restriction site and an antisense primer comprising another restriction site, e.g. a non-identical restriction site to facilitate cloning. The same is repeated for the isolated nucleic acid encoding an antigen. Ligation of the truncated ActA and antigen sequences and insertion into a plasmid or vector produces a vector encoding truncated ActA joined to a terminus of the antigen. The two molecules are joined either directly or by a short spacer introduced by the restriction site.

Fusion proteins comprising an antigen or immunogenic fragment thereof may be prepared by any suitable method, including, for example, cloning and restriction of appropriate sequences or direct chemical synthesis. Alternatively, subsequences may be cloned and the appropriate subsequences cleaved using appropriate restriction enzymes. The fragments may then be ligated to produce the desired DNA sequence. In one embodiment, DNA encoding the antigen can be produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now fondling the carboxyl sequence). The antigen is ligated into a plasmid.

In one embodiment, a plasmid provided herein is stably maintained inside a host cell, including a host Listeria cell. “Stably maintained” refers to maintenance of a nucleic acid molecule or plasmid in the absence of selection (e.g. antibiotic selection) for at least 10 generations, without detectable loss. In another embodiment, the period is 15 generations, 20-30 generations, 40-50 generations, 60-80 generations, 100-200 generations, or 200-500 generations. In another embodiment, the period is more than 500 generations. In another embodiment, the nucleic acid molecule or plasmid is maintained stably in vitro (e.g. in culture). In another embodiment, the nucleic acid molecule or plasmid is maintained stably in vivo. In another embodiment, the nucleic acid molecule or plasmid is maintained stably both in vitro and in vitro.

In one embodiment, the recombinant Listeria strain of methods and compositions provided herein is a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of another Listeria species.

In another embodiment, a recombinant Listeria strain of the present invention has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the Listeria strain contains a genomic insertion of the gene encoding the antigen-containing recombinant peptide. In another embodiment, the Listeria strain carries a plasmid comprising the gene encoding the antigen-containing recombinant peptide. In another embodiment, the passaging is performed as described herein. In another embodiment, the passaging is performed by other methods known in the art.

In another embodiment, inducible and tissue specific expression of the nucleic acid encoding a peptide of the present invention is accomplished by placing the nucleic acid encoding the peptide under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In another embodiment, a promoter that is induced in response to inducing agents such as metals, glucocorticoids, and the like, is utilized. Thus, it will be appreciated that the invention includes the use of any promoter/regulatory sequence, which is either known or unknown, and which is capable of driving expression of the desired protein operably linked thereto.

It will be appreciated by a skilled artisan that the tetra “heterologous” encompasses a nucleic acid, amino acid, peptide, polypeptide, or protein derived from a different species than the reference species. Thus, for example, a Listeria strain expressing a heterologous polypeptide, in one embodiment, would express a polypeptide that is not native or endogenous to the Listeria strain, or in another embodiment, a polypeptide that is not normally expressed by the Listeria strain, or in another embodiment, a polypeptide from a source other than the Listeria strain. In another embodiment, heterologous may be used to describe something derived from a different organism within the same species. In another embodiment, the heterologous antigen is expressed by a recombinant strain of Listeria, and is processed and presented to cytotoxic T-cells upon infection of mammalian cells by the recombinant strain. In another embodiment, the heterologous antigen expressed by Listeria species need not precisely match the corresponding unmodified antigen or protein in the tumor cell or infectious agent so long as it results in a T-cell response that recognizes the unmodified antigen or protein which is naturally expressed in the mammal. The term heterologous antigen may be referred to herein as “antigenic polypeptide”, “heterologous protein”, “heterologous protein antigen”, “protein antigen”, “antigen”, and the like.

In one embodiment, the two molecules of a fusion protein provided herein are joined directly. In another embodiment, the two molecules are joined by a short spacer peptide, consisting of one or more amino acids. In one embodiment, the spacer has no specific biological activity other than to join the proteins or to preserve some minimum distance or other spatial relationship between them. In another embodiment, the constituent amino acids of the spacer are selected to influence some property of the molecule such as the folding, net charge, or hydrophobicity. In another embodiment, the two molecules of the protein (for example, the truncated ActA fragment and the antigen) are synthesized separately or unfused. In another embodiment, the two molecules of the protein are synthesized separately from the same nucleic acid. In yet another embodiment, the two molecules are individually synthesized from separate nucleic acids.

It will be appreciated by a skilled artisan that the term “Administration” and “treatment,” as it applies to an animal, human, experimental subject, cell, tissue, organ, or biological fluid, may encompass contacting of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also may encompass in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, rabbit) and most preferably a human.

It will be appreciated by a skilled artisan that the term “pharmaceutical composition” may encompass a therapeutically effective amount of the active ingredient or ingredients comprising the Listeria strain, with a pharmaceutically acceptable carrier or diluent. The term “pharmaceutical composition” may be used interchangeably herein with the terms “composition,” “immunogenic composition,” “medicament,” or “vaccine”.

It will be appreciated by a skilled artisan that the terms “therapeutically effective amount”, in reference to the treatment of a disease, wherein in one embodiment the disease is a tumor or cancer and in such cases may encompass an amount capable of invoking one or more of the following effects: (1) inhibition, to some extent, of tumor growth, including, slowing down and complete growth arrest; (2) reduction in the number of tumor cells; (3) reduction in tumor size; (4) inhibition (i.e., reduction, slowing down or complete stopping) of tumor cell infiltration into peripheral organs; (5) inhibition (i.e., reduction, slowing down or complete stopping) of metastasis; (6) enhancement of anti-tumor immune response, which may, but does not have to, result in the regression or rejection of the tumor; and/or (7) relief, to some extent, of one or more symptoms associated with the disorder. A “therapeutically effective amount” of a pharmaceutical composition or vaccine provided herein for purposes of treatment of tumor may be determined empirically and in a routine manner.

It will be appreciated by a skilled artisan that the terms “immunogenic composition,” “composition” and “pharmaceutical composition” may be used interchangeably. It is also to be understood that administration of such compositions enhances an immune response, or increase a T effector cell to regulatory T cell ratio or elicit an anti-tumor immune response, amongst other effects, as further provided herein.

In another embodiment, an immune response elicited by the methods and compositions provided herein comprises an immune response to at least one subdominant epitope of the antigen and/or at least one dominant epitope of the antigen. In another embodiment, the dominant epitope or subdominant epitope is dominant or subdominant, respectively, in the subject being treated. In another embodiment, the dominant epitope or subdominant epitope is dominant or subdominant in a population being treated.

In another embodiment, administration of compositions of the present invention increase the number of antigen-specific T cells, activates co-stimulatory receptors on T cells, induces proliferation of memory and/or effector T cells, increases proliferation of T cells, and/or negates tumor immunosuppressive signaling.

Compositions of this invention may be used in methods of this invention in order to elicit an enhanced anti-tumor T cell response in a subject, in order to inhibit tumor-mediated immunosuppression in a subject, or for increasing the ratio or T effector cells to regulatory T cells (T_{regs}) in the spleen and tumor of a subject, or any combination thereof.

In one embodiment, the compositions provided herein may be used in combination with (either concurrently, prior to, or following) an administration of an additional therapeutic modality. It will be appreciated by a skilled artisan that additional therapeutic modalities may encompass surgery (e.g. to remove a tumor), radiation therapy, chemotherapy, or a combination thereof.

Examples of chemotherapeutic agents include alkyl ating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gamma1I and calicheamicin phiI 1 , see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

It will be appreciated by a skilled artisan that the term “Chemotherapeutic agent” may encompass a chemical or biological substance that can cause death of cancer cells, or interfere with growth, division, repair, and/or function of cancer cells. Classes of chemotherapeutic agents include, but are not limited to: alkylating agents, antimetabolites, kinase inhibitors, spindle poison plant alkaloids, cytoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, anti-sense oligonucleotides that inhibit expression of genes implicated in abnormal cell proliferation or tumor growth. Chemotherapeutic agents useful in the treatment methods of the present invention include cytostatic and/or cytotoxic agents.

Each therapeutic agent in a combination therapy provided herein may be administered either alone or in a medicament (also referred to herein as a pharmaceutical composition) which comprises the therapeutic agent and one or more pharmaceutically acceptable carriers, excipients and diluents, according to standard pharmaceutical practice.

It will be appreciated by a skilled artisan that the term “pharmaceutically acceptable carrier” may encompass any inactive substance that is suitable for use in a formulation for the administration to a subject of a live-attenuated Listeria strain that is used to stimulate antigen-presenting cells (APCs) capable of driving a cellular immune response to an antigen or fragment thereof expressed by a disease cell (e.g. a tumor cell).

It will be appreciated by a skilled artisan that the term “Transforming,” may encompass engineering a bacterial cell to take up a plasmid or other heterologous DNA molecule. In another embodiment, “transforming” refers to engineering a bacterial cell to express a gene of a plasmid or other heterologous DNA molecule. Each possibility represents a separate embodiment of the methods and compositions as provided herein.

### II. Therapeutic Methods of Use

In one embodiment, the present invention provides an immunogenic composition as described above, for treating cancer. For example, an immunogenic composition used in a method provided herein comprises a Listeria strain expressing a fusion polypeptide as described throughout, wherein the fusion polypeptide comprises an antigen or fragment thereof.

It will be appreciated by a skilled artisan that the term “treating” may encompass curing a disease. In another embodiment, “treating” may encompass preventing a disease. In another embodiment, “treating” may encompass reducing the incidence of a disease. In another embodiment, “treating” may encompass ameliorating symptoms of a disease. In another embodiment, “treating” may encompass increasing performance free survival or overall survival of a patient. In another embodiment, “treating” may encompass stabilizing the progression of a disease. In another embodiment, “treating” may encompass inducing remission. In another embodiment, “treating” may encompass slowing the progression of a disease. The terms “reducing”, “suppressing” and “inhibiting” refer to lessening or decreasing.

It will also be appreciated by a skilled artisan that the ten. “treating” may encompass both therapeutic treatment and prophylactic or preventative measures, wherein the object is to prevent or lessen the targeted pathologic condition or disorder as described herein. Thus, in some embodiments, treating may include directly affecting or curing, suppressing, inhibiting, preventing, reducing the severity of, delaying the onset of, reducing symptoms associated with the disease, disorder or condition, or a combination thereof. Thus, in other embodiments, “treating” may encompass inter alia delaying progression, expediting remission, inducing remission, augmenting remission, speeding recovery, increasing efficacy of or decreasing resistance to alternative therapeutics, or a combination thereof.

It will be appreciated by a skilled artisan that the terms “preventing” or “impeding” may encompass, inter alia, delaying the onset of symptoms, preventing relapse to a disease, decreasing the number or frequency of relapse episodes, increasing latency between symptomatic episodes, or a combination thereof. It will also be appreciated by a skilled artisan that the terms “suppressing” or “inhibiting”, may encompass, inter alia, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, symptoms are primary, while in another embodiment, symptoms are secondary. It will be appreciated by a skilled artisan that the term “primary” refers to a symptom that is a direct result of a particular disease or disorder, while the term “secondary” refers to a symptom that is derived from or consequent to a primary cause. In one embodiment, the compounds for use in the present invention treat primary or secondary symptoms or secondary complications. In another embodiment, “symptoms” may be any manifestation of a disease or pathological condition.

In one embodiment, the immunogenic compositions provided herein are useful for preventing, suppressing, inhibiting, or treating an autoimmune disease. In one embodiment, the autoimmune disease is any autoimmune disease known in the art, including, but not limited to, a rheumatoid arthritis (RA), insulin dependent diabetes mellitus (Type 1 diabetes), multiple sclerosis (MS), Crohn's disease, systemic lupus erythematosus (SLE), scleroderma, Sjogren's syndrome, pemphigus vulgaris, pemphigoid, addison's disease, ankylosing spondylitis, aplastic anemia, autoimmune hemolytic anemia, autoimmune hepatitis, coeliac disease, dermatomyositis, Goodpasture's syndrome, Graves' disease, Guillain-Barre syndrome, Hashimoto's disease, idiopathic leucopenia, idiopathic thrombocytopenic purpura, male infertility, mixed connective tissue disease, myasthenia gravis, pernicious anemia, phacogenic uveitis, primary biliary cirrhosis, primary myxoedema, Reiter's syndrome, stiff man syndrome, thyrotoxicosis, ulceritive colitis, and Wegener's granulomatosis. In another embodiment, the invention is also drawn to the agonist antibody directed against ICOS according to the invention or a derivative thereof for use for treating an inflammatory disorder selected in the group consisting of inflammatory disorder of the nervous system such as multiple sclerosis, mucosal inflammatory disease such as inflammatory bowel disease, asthma or tonsillitis, inflammatory skin disease such as dermatitis, psoriasis or contact hypersensitivity, and autoimmune arthritis such as rheumatoid arthritis.

In one embodiment, a disease described herein is a cancer or a tumor (solid or not). It will be understood by a skilled artisan that an illustrative example may be a breast cancer, a cervical cancer, an Her2 containing cancer, a melanoma, a pancreatic cancer, an ovarian cancer, a gastric cancer, a carcinomatous lesion of the pancreas, a pulmonary adenocarcinoma, a glioblastoma multiforme, a colorectal adenocarcinoma, a pulmonary squamous adenocarcinoma, a gastric adenocarcinoma, an ovarian surface epithelial neoplasm (e.g. a benign, proliferative or malignant variety thereof), an oral squamous cell carcinoma, an endometrial carcinoma, a bladder cancer, a head and neck cancer, a prostate carcinoma, a oropharyngeal cancer, a lung cancer, an anal cancer, a colorectal cancer, an esophageal cancer, a mesothelioma, a sarcoma, a leukemia, a lymphoma (including a B-cell lymphoma) or any combination thereof.

In another embodiment, the cancer being treated is breast cancer, a central nervous system (CNS) cancer, a head and neck cancer, an osteosarcoma (OSA), a canine osteosarcoma (OSA), or Ewing's sarcoma (ES). In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non-small-cell lung carcinoma. In another embodiment, the cancer is a CNS carcinoma. In another embodiment, the cancer is an endometrial carcinoma. In another embodiment, the cancer is a bladder cancer. In another embodiment, the cancer is mesothelioma. In another embodiment, the cancer is malignant mesothelioma (MM). In another embodiment, the cancer is a melanoma. In another embodiment, the cancer is a glioma. In another embodiment, the cancer is a germ cell tumor. In another embodiment, the cancer is a choriocarcinoma. In another embodiment, the cancer is a lymphoma, leukemia, myeloma or any survivin-expressing cancer known in the art.

In another embodiment, the cancer is a pancreatic carcinoma. In another embodiment, the cancer is pancreatic ductal carcinoma. In another embodiment, the cancer is acinar cell carcinoma of the pancreas, or cystadenocarcinoma. In another embodiment, the cancer is pancreatic neuroendocrine tumor. In another embodiment, the cancer is insulinoma or gastrinoma. In another embodiment, the cancer is any prostate carcinoma known in the art.

In another embodiment, the cancer is refractory. In another embodiment, the cancer is advanced. In another embodiment, the cancer is a metastasis. In another embodiment, a cancer or solid tumor is a result of relapse or metastatic disease.

In another embodiment, cells of a tumor that is targeted by the methods and compositions of the present invention express a tumor antigen or a fragment thereof. In another embodiment, cells of the tumor that is targeted by methods and compositions of the present invention express low levels of MHC.

In one embodiment, cancer or tumors may be prevented in specific populations known to be susceptible to a particular cancer or tumor. In one embodiment, such susceptibilty may be due to environmental factors, such as smoking, which in one embodiment, may cause a population to be subject to lung cancer, while in another embodiment, such susceptibility may be due to genetic factors, for example a population with BRCA1/2 mutations may be susceptible, in one embodiment, to breast cancer, and in another embodiment, to ovarian cancer. In another embodiment, one or more mutations on chromosome 8q24, chromosome 17q12, and chromosome 17q24.3 may increase susceptibility to pancreatic cancer, as is known in the art. Other genetic and environmental factors contributing to cancer susceptibility are known in the art.

Following the administration of an immunogenic composition provided herein, the methods provided herein induce the expansion of T effector cells in peripheral lymphoid organs leading to an enhanced presence of T effector cells at the tumor site. In another embodiment, the methods provided herein induce the expansion of T effector cells in peripheral lymphoid organs leading to an enhanced presence of T effector cells at the periphery. Such expansion of T effector cells leads to an increased ratio of T effector cells to regulatory T cells in the periphery and at the tumor site without affecting the number of Tregs. It will be appreciated by the skilled artisan that peripheral lymphoid organs include, but are not limited to, the spleen, peyer's patches, the lymph nodes, the adenoids, etc. In one embodiment, the increased ratio of T effector cells to regulatory T cells occurs in the periphery without affecting the number of Tregs. In another embodiment, the increased ratio of T effector cells to regulatory T cells occurs in the periphery, the lymphoid organs and at the tumor site without affecting the number of Tregs at these sites. In another embodiment, the increased ratio of T effector cells decreases the frequency of Tregs, but not the total number of Tregs at these sites.

In one embodiment, provided herein are methods of eliciting an enhanced immune response against a disease in a subject, the method comprising the step of administering to the subject a composition comprising a recombinant Listeria provided herein. In another embodiment, provided herein are methods of eliciting an enhanced immune response against a tumor or cancer in a subject, the method comprising the step of administering to the subject a composition comprising a recombinant Listeria provided herein.

In one embodiment, provided herein is a method of preventing a disease in a subject the method comprising the step of administering to the subject a composition comprising a recombinant Listeria provided herein. In another embodiment, provided herein is a method of preventing a tumor or cancer in a subject the method comprising the step of administering to the subject a composition comprising a recombinant Listeria provided herein. In another embodiment, the administration of Listeria expressing a fusion protein with a truncated ActA elicits an immune response against other tumor-associated antigens as a result of epitope spreading.

In one embodiment, provided herein is a method of treating a disease in a subject, the method comprising the step of administering to the subject a composition comprising a recombinant Listeria provided herein. In another embodiment, provided herein is a method of treating a tumor or cancer in a subject, the method comprising the step of administering to the subject a composition comprising a recombinant Listeria provided herein.

In one embodiment, provided herein is a method of delaying the progression of a disease in a subject, the method comprising the step of administering to the subject a composition comprising a recombinant Listeria provided herein. In another embodiment, provided herein is a method of delaying the progression of a tumor or cancer in a subject, the method comprising the step of administering to the subject a composition comprising a recombinant Listeria provided herein.

In one embodiment, provided herein is a method of prolonging the survival of a subject having disease, the method comprising the step of administering to the subject a composition comprising a recombinant Listeria provided herein. In one embodiment, provided herein is a method of prolonging the survival of a subject having tumor or cancer, the method comprising the step of administering to the subject

In one embodiment, provided herein is a method of inhibiting, impeding, or delaying metastatic tumor or cancer in a subject having a disease, the method comprising the step of administering to the subject a composition comprising a recombinant Listeria provided herein.

In one embodiment, provided herein is a method of inducing an anti-disease immune response in a subject, the method comprising the step of administering to the subject a composition comprising a recombinant Listeria provided herein. In another embodiment, provided herein is a method of inducing an anti-tumor or anti-cancer immune response in a subject, the method comprising the step of administering to the subject a composition comprising a recombinant Listeria provided herein.

In one embodiment, provided herein is a method of augmenting an anti-tumor or anti-cancer immune response in a subject, the method comprising the step of administering to the subject a composition comprising a recombinant Listeria provided herein.

In one embodiment, provided herein is a method of preventing an escape mutation in the treatment of a cancer, the method comprising the step of administering to the subject a composition comprising a recombinant Listeria provided herein.

In one embodiment, provided herein is a method of inducing regression of a tumor or cancer in a subject, the method comprising the step of administering to the subject a composition comprising a recombinant Listeria provided herein.

In one embodiment, provided herein is a method of decreasing the frequency of intra-tumoral T regulatory cells, the method comprising the step of administering to the subject a composition comprising a recombinant Listeria provided herein.

In one embodiment, provided herein is a method of treating a metastatic disease coming from an antigen-expressing tumor in a subject, the method comprising the step of administering to the subject a composition comprising a recombinant Listeria provided herein.

In one embodiment, provided herein is a method of breaking tolerance in a subject to a self-antigen-expressing tumor or cancer in the subject, the method comprising the step of administering to the subject a composition comprising a recombinant Listeria provided herein.

In one embodiment, provided herein is a method of impeding growth of a tumor or cancer in a subject, the method comprising the step of administering to the subject a composition comprising a recombinant Listeria provided herein.

In another embodiment, the methods provided herein of inducing an anti-disease, or anti-tumor or anti-cancer immune response allow treating a disease or tumor or cancer, respectively, in a subject.

In one embodiment, the present invention provides a method for “epitope spreading” of an anti-tumor response. In another embodiment, the immunization using the compositions and methods provided herein induce epitope spreading onto other tumors bearing antigens other than the antigen carried in the vaccine or compositions provided herein.

In one embodiment, an immune response provided herein is a cell mediated anti-tumor immune response. In one embodiment, a cell mediated immune response is a CD8+ T cell response or a CD4+ T cell response or a natural killer (NK) cell response. It will be appreciated by a skilled artisan that a cell-mediated response may encompass a CD8+ T cell response or a CD4+ T cell response or a natural killer (NK) cell response, or any combination thereof.

In one embodiment, provided herein is a method of treating, suppressing, or inhibiting a cancer or a tumor growth in a subject by epitope spreading wherein the cancer is associated with expression of an antigen or fragment thereof comprised in a composition provided herein. In another embodiment, the subject mounts an immune response against the antigen-expressing cancer or the antigen-expressing tumor, thereby treating, suppressing, or inhibiting a cancer or a tumor growth in a subject.

In one embodiment, provided herein is a method of increasing a ratio of T effector cells to regulatory T cells (Tregs) in the spleen and tumor microenvironments of a subject, comprising administering an immunogenic composition comprising a recombinant Listeria provided herein. In another embodiment, increasing a ratio of T effector cells to regulatory T cells (Tregs) in the spleen and tumor microenvironments in a subject allows for a more potent anti-tumor response in the subject.

It will be understood by a skilled artisan that T effector cells may comprise CD4+FoxP3-T cells, and CD8+ T cells. In one embodiment, a regulatory T cells is a CD4+FoxP3+T cell.

In one embodiment, the present invention provides a method of preventing or treating a tumor or cancer in a human subject, comprising the step of administering to the subject a composition comprising a recombinant Listeria provided herein, the recombinant Listeria strain comprising a recombinant polypeptide comprising an N-terminal fragment of an ActA protein and tumor-associated antigen, whereby the recombinant Listeria strain induces an immune response against the tumor-associated antigen, thereby treating a tumor or cancer in a human subject. In another embodiment, the immune response is a T-cell response. It will be understood by a skilled artisan that a T-cell response may be a CD4+FoxP3− T cell response or a CD8+ T cell response or a combination thereof.

In another embodiment, the present invention provides a method of inducing regression of a tumor in a subject, comprising the step of administering to the subject a composition comprising a recombinant Listeria provided herein. In another embodiment, the present invention provides a method of reducing the incidence or relapse of a tumor or cancer, comprising the step of administering to the subject a composition comprising a recombinant Listeria provided herein. In another embodiment, the present invention provides a method of suppressing the formation of a tumor in a subject, comprising the step of administering to the subject the a composition comprising a recombinant Listeria provided herein. In another embodiment, the present invention provides a method of inducing a remission of a cancer in a subject, comprising the step of administering to the subject a composition comprising a recombinant Listeria provided herein. In another embodiment, the present invention provides a method of extending a remission of a tumor or cancer in a subject, comprising the step of administering to the subject a composition comprising a recombinant Listeria provided herein. In another embodiment, the present invention provides a method of reducing the size of a tumor a subject, comprising the step of administering to the subject a composition comprising a recombinant Listeria provided herein.

In another embodiment, a method of treating reduces tumor size. Reduction of tumor size may be partial or complete. In another embodiment, methods of this invention reduce tumor size by 90%. In another embodiment, methods of this invention reduce tumor size by 80%. In another embodiment, methods reduce tumor size by 70%. In another embodiment, methods reduce tumor size by 60%. In another embodiment, methods reduce tumor size by 50%.

In one embodiment, a method of treating increases the time to disease progression. In one embodiment, the time to disease progression is increased by at least 2 months as compared to an untreated subject. In one embodiment, the time to disease progression is increased by at least 4 months as compared to an untreated subject. In another embodiment, the time to disease progression is increased by at least 6 months as compared to an untreated subject. In one embodiment, the time to disease progression is increased by at least 1 year as compared to an untreated subject. In one embodiment, the time to disease progression is increased by at least 2 years as compared to an untreated subject. In one embodiment, the time to disease progression is increased by at least 3 years as compared to an untreated subject. In another embodiment, the time to disease progression is increased by at least 4 years as compared to an untreated subject. In one embodiment, the time to disease progression is increased by at least 5 years as compared to an untreated subject.

In another embodiment, the present invention provides a method of impeding a growth of an antigen-expressing cancer in a subject, comprising administering to the subject a composition comprising a recombinant Listeria provided herein, wherein the recombinant polypeptide comprising an N-terminal fragment of a ActA protein fused to an antigen, and wherein the antigen has one or more subdominant CD8+ T cell epitopes. In another embodiment, the antigen does contain the dominant CD8+ T cell epitopes.

“Dominant CD8+ T cell epitope” refers to an epitope that is recognized by over 30% of the antigen-specific CD8+ T cells that are elicited by vaccination, infection, or a malignant growth with a protein or a pathogen or cancer cell containing the protein. In another embodiment, the term refers to an epitope recognized by over 35% of the antigen-specific CD8+ T cells that are elicited thereby. In another embodiment, the term refers to an epitope recognized by over 40% of the antigen-specific CD8+ T cells. In another embodiment, the term refers to an epitope recognized by over 45% of the antigen-specific CD8+ T cells. In another embodiment, the term refers to an epitope recognized by over 50% of the antigen-specific CD8+ T cells. In another embodiment, the term refers to an epitope recognized by over 55% of the antigen-specific CD8+ T cells. In another embodiment, the term refers to an epitope recognized by over 60% of the antigen-specific CD8+ T cells. In another embodiment, the term refers to an epitope recognized by over 65% of the antigen-specific CD8+ T cells. In another embodiment, the term refers to an epitope recognized by over 70% of the antigen-specific CD8+ T cells. In another embodiment, the term refers to an epitope recognized by over 75% of the antigen-specific CD8+ T cells. In another embodiment, the term refers to an epitope recognized by over 80% of the antigen-specific CD8+ T cells. In another embodiment, the term refers to an epitope recognized by over 85% of the antigen-specific CD8+ T cells. In another embodiment, the term refers to an epitope recognized by over 90% of the antigen-specific CD8+ T cells. In another embodiment, the term refers to an epitope recognized by over 95% of the antigen-specific CD8+ T cells. In another embodiment, the term refers to an epitope recognized by over 96% of the antigen-specific CD8+ T cells. In another embodiment, the term refers to an epitope recognized by over 97% of the antigen-specific CD8+ T cells. In another embodiment, the term refers to an epitope recognized by over 98% of the antigen-specific CD8+ T cells.

“Subdominant CD8+ T cell epitope” refers to an epitope recognized by fewer than 30% of the antigen-specific CD8+ T cells that are elicited by vaccination, infection, or a malignant growth with a protein or a pathogen or cancer cell containing the protein. In another embodiment, the term refers to an epitope recognized by fewer than 28% of the antigen-specific CD8+ T cells. In another embodiment, the term refers to an epitope recognized by over 26% of the antigen-specific CD8+ T cells. In another embodiment, the term refers to an epitope recognized by fewer than 24% of the antigen-specific CD8+ T cells. In another embodiment, the term refers to an epitope recognized by over 22% of the antigen-specific CD8+ T cells. In another embodiment, the term refers to an epitope recognized by fewer than 20% of the antigen-specific CD8+ T cells. In another embodiment, the term refers to an epitope recognized by over 18% of the antigen-specific CD8+ T cells. In another embodiment, the term refers to an epitope recognized by fewer than 16% of the antigen-specific CD8+ T cells. In another embodiment, the term refers to an epitope recognized by over 14% of the antigen-specific CD8+ T cells. In another embodiment, the teixii refers to an epitope recognized by over 12% of the antigen-specific CD8+ T cells. In another embodiment, the term refers to an epitope recognized by fewer than 10% of the antigen-specific CD8+ T cells. In another embodiment, the term refers to an epitope recognized by over 8% of the antigen-specific CD8+ T cells. In another embodiment, the term refers to an epitope recognized by fewer than 6% of the antigen-specific CD8+ T cells. In another embodiment, the term refers to an epitope recognized by fewer than 5% of the antigen-specific CD8+ T cells. In another embodiment, the term refers to an epitope recognized by over 4% of the antigen-specific CD8+ T cells. In another embodiment, the term refers to an epitope recognized by fewer than 3% of the antigen-specific CD8+ T cells. In another embodiment, the term refers to an epitope recognized by fewer than 2% of the antigen-specific CD8+ T cells. In another embodiment, the term refers to an epitope recognized by fewer than 1% of the antigen-specific CD8+ T cells. In another embodiment, the term refers to an epitope recognized by fewer than 0.5% of the antigen-specific CD8+ T cells.

In one embodiment, vaccination with recombinant Listeria expressing the ActA-antigen fusions provided herein induces epitope spreading.

The antigen in methods and compositions of the present invention is, in one embodiment, expressed at a detectable level on a non-tumor cell of the subject. In another embodiment, the antigen is expressed at a detectable level on at least a certain percentage (e.g. 0.01%, 0.03%, 0.1%, 0.3%, 1%, 2%, 3%, or 5%) of non-tumor cells of the subject. In one embodiment, “non-tumor cell” refers to a cell outside the body of the tumor. In another embodiment, “non-tumor cell” refers to a non-malignant cell. In another embodiment, “non-tumor cell” refers to a non-transfornied cell. In another embodiment, the non-tumor cell is a somatic cell. In another embodiment, the non-tumor cell is a germ cell.

“Detectable level” refers, in one embodiment, to a level that is detectable when using a standard assay. In one embodiment, the assay is an immunological assay. In one embodiment, the assay is enzyme-linked immunoassay (ELISA). In another embodiment, the assay is Western blot. In another embodiment, the assay is FACS. In yet another embodiment, the assay is Western blot. In another embodiment, the assay is PCR. It is to be understood by a skilled artisan that other assays available in the art can be used in the methods provided herein. In another embodiment, a detectable level is determined relative to the background level of a particular assay. Methods for performing each of these techniques are well known to those skilled in the art.

In another embodiment, the present invention provides a method for inducing formation of anti-cancer cytotoxic T cells in a host having cancer, comprising administering to the host composition comprising a recombinant Listeria provided herein, thereby inducing formation of cytotoxic T cells in a host having cancer.

In one embodiment, provided herein is a method of administering a composition of the present invention. In another embodiment, provided herein is a method of administering a vaccine of the present invention. In another embodiment, provided herein is a method of administering a recombinant polypeptide or recombinant nucleotide of the present invention. In another embodiment, the step of administering a composition, recombinant polypeptide or recombinant nucleotide of the present invention is performed with an attenuated recombinant faun of Listeria comprising a recombinant nucleotide or expressing a recombinant polypeptide. In another embodiment, the administering is performed with a DNA vaccine (e.g. a naked DNA vaccine). In another embodiment, administration of a recombinant polypeptide of the present invention is performed by producing the protein recombinantly, then administering the recombinant protein to a subject.

In one embodiment, a composition is administered to the cells of the subject ex vivo; in another embodiment, the composition is administered to the cells of a donor ex vivo; in another embodiment, the composition is administered to the cells of a donor in vivo, and then is transferred to the subject.

Various embodiments of dosage ranges are contemplated by this invention.

In one embodiment, the dose of the attenuated Listeria strain comprised by the immunogenic composition provided herein is administered to a subject at a dose of 1×10⁷−3.31×10¹⁰ colony forming units (CFU). In another embodiment, the dose is 1×10⁸−3.31×10¹⁰ CFU. In another embodiment, the dose is 1×10⁹−3.31×10¹⁰ CFU. In another embodiment, the dose is 3-5×10⁹ CFU.

In another embodiment, the dose is 1×10⁷ organisms. In another embodiment, the dose is 1×10⁸ organisms. In another embodiment, the dose is 1×10⁹ organisms. In another embodiment, the dose is 1.5×10⁹ organisms. In another embodiment, the dose is 2×10⁹ organisms. In another embodiment, the dose is 3×10⁹ organisms. In another embodiment, the dose is 4×10⁹ organisms. In another embodiment, the dose is 5×10⁹ organisms. In another embodiment, the dose is 6×10⁹ organisms. In another embodiment, the dose is 7×10⁹ organisms. In another embodiment, the dose is 8×10⁹ organisms. In another embodiment, the dose is 10×10⁹ organisms. In another embodiment, the dose is 1.5×10¹⁰ organisms. In another embodiment, the dose is 2×10¹⁰ organisms. In another embodiment, the dose is 2.5×10¹⁰ organisms. In another embodiment, the dose is 3×10¹⁰ organisms. In another embodiment, the dose is 3.3×10¹⁰ organisms. In another embodiment, the dose is 4×10¹⁰ organisms. In another embodiment, the dose is 5×10¹⁰ organisms.

In one embodiment, repeat administrations (doses) of compositions provided herein may be undertaken immediately following the first course of treatment or after an interval of days, weeks or months to achieve tumor regression. In another embodiment, repeat doses may be undertaken immediately following the first course of treatment or after an interval of days, weeks or months to achieve suppression of tumor growth. Assessment may be determined by any of the techniques known in the art, including diagnostic methods such as imaging techniques, analysis of serum tumor markers, biopsy, or the presence, absence or amelioration of tumor associated symptoms.

In one embodiment, the methods of the present invention further comprise the step of administering to the subject a booster vaccination. It will be appreciated by the skilled artisan that the term “Boosting” may encompass administering an additional strain or immunogenic composition or recombinant Listeria strain dose or immune checkpoint inhibitor alone or in combination to a subject. In another embodiment, of methods of the present invention, 2 boosts (or a total of 3 inoculations) are administered. In another embodiment, 3 boosts are administered. In another embodiment, 4 boosts are administered. In another embodiment, 5 boosts are administered. In another embodiment, 6 boosts are administered. In another embodiment, more than 6 boosts are administered.

In another embodiment, a method of present invention further comprises the step of boosting the subject with a recombinant Listeria strain provided herein. In another embodiment, the recombinant Listeria strain used in the booster inoculation is the same as the strain used in the initial “priming” inoculation. In another embodiment, the booster strain is different from the priming strain. In another embodiment, the same doses are used in the priming and boosting inoculations. In another embodiment, a larger dose is used in the booster. In another embodiment, a smaller dose is used in the booster. In one embodiment, the booster vaccination follows a single priming vaccination. In another embodiment, a single booster vaccination is administered after the priming vaccinations. In another embodiment, two booster vaccinations are administered after the priming vaccinations. In another embodiment, three booster vaccinations are administered after the priming vaccinations. In one embodiment, the period between a prime and a boost strain is experimentally determined by the skilled artisan. In another embodiment, the period between a prime and a boost strain is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost strain is administered 8-10 weeks after the prime strain.

Heterologous “prime boost” strategies have been effective for enhancing immune responses and protection against numerous pathogens. Schneider et al., Immunol. Rev. 170:29-38 (1999); Robinson, H. L., Nat. Rev. Immunol. 2:239-50 (2002); Gonzalo, R. M. et al., Strain 20:1226-31 (2002); Tanghe, A., Infect. Immun. 69:3041-7 (2001). Providing antigen in different forms in the prime and the boost injections appears to maximize the immune response to the antigen. DNA strain priming followed by boosting with protein in adjuvant or by viral vector delivery of DNA encoding antigen appears to be the most effective way of improving antigen specific antibody and CD4+ T-cell responses or CD8+ T-cell responses respectively. Shiver J. W. et al., Nature 415: 331-5 (2002); Gilbert, S. C. et al., Strain 20:1039-45 (2002); Billaut-Mulot, O. et al., Strain 19:95-102 (2000); Sin, J. I. et al., DNA Cell Biol. 18:771-9 (1999). Recent data from monkey vaccination studies suggests that adding CRL1005 poloxamer (12 kDa, 5% POE), to DNA encoding the HIV gag antigen enhances T-cell responses when monkeys are vaccinated with an HIV gag DNA prime followed by a boost with an adenoviral vector expressing HIV gag (Ad5-gag). The cellular immune responses for a DNA/poloxamer prime followed by an Ad5-gag boost were greater than the responses induced with a DNA (without poloxamer) prime followed by Ad5-gag boost or for Ad5-gag only. Shiver, J. W. et al. Nature 415:331-5 (2002). U.S. Patent Appi. Publication No. US 2002/0165172 Al describes simultaneous administration of a vector construct encoding an immunogenic portion of an antigen and a protein comprising the immunogenic portion of an antigen such that an immune response is generated. The document is limited to hepatitis B antigens and HIV antigens. Moreover, U.S. Pat. No. 6,500,432 is directed to methods of enhancing an immune response of nucleic acid vaccination by simultaneous administration of a polynucleotide and polypeptide of interest. According to the patent, simultaneous administration means administration of the polynucleotide and the polypeptide during the same immune response, preferably within 0-10 or 3-7 days of each other. The antigens contemplated by the patent include, among others, those of Hepatitis (all forms), HSV, HIV, CMV, EBV, RSV, VZV, HPV, polio, influenza, parasites (e.g., from the genus Plasmodium), and pathogenic bacteria (including but not limited to M. tuberculosis, M. leprae, Chlamydia, Shigella, B. burgdorferi, enterotoxigenic E. coli, S. typhosa, H. pylori, V. cholerae, B. pertussis, etc.). All of the above references are herein incorporated by reference in their entireties.

In one embodiment, a composition comprising a recombinant Listeria provided herein is administered in combination with an adjuvant. It will be appreciated by a skilled artisan that an adjuvant may include, but not be limited to, any of the following: a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

In one embodiment, a composition comprising a recombinant Listeria provided herein is administered as a combination therapy with an immunosuppressive molecule antagonist in order to stimulate APCs capable of driving a cellular immune response to antigen expressing cells. It will be understood by a skilled artisan that such a combination therapy may be administered in a single dosage form. In some instances, the immunosuppressive molecule antagonist and a composition comprising a live-attenuated Listeria are administered in separate dosage forms.

In one embodiment, administration of an immunosuppressive molecule antagonist and a live-attenuated Listeria strain provided herein is maintained throughout a period of treatment or prevention. In another embodiment, anti-cancer activity is achieved by subsequent administration of either component in isolation, i.e.—the immunosuppressive molecule antagonist or the live-attenuated Listeria strain (or a composition comprising either component).

It will be well appreciated by a skilled artisan that the terms “immunosuppressive antagonist” and “immune checkpoint inhibitor” may be used interchangeably herein, both of which may function to inhibit, down-regulate or suppress T-effector cell function in response to a disease, including a tumor or cancer. Immunosuppressive molecules are known in the art and include but are not limited to inhibitor is a Programmed Death 1 (PD-1) signaling pathway inhibitor, CD80/86 signaling pathway inhibitor, a CTLA-4, Inhibitor T cell membrane protein 3 (TIM3), adenosine A2a receptor (A2aR) and lymphocyte activation gene 3 (LAG3), killer immunoglobulin receptor (KIR) or cytotoxic T-lymphocyte antigen-4 (CTLA-4). In another embodiment, the checkpoint inhibitor protein is one belonging to the B7/CD28 receptor superfamily. In another embodiment, an immune checkpoint inhibitor is any other antigen-presenting cell :T-cell signaling pathway inhibitor known in the art.

In another embodiment, the PD-1 signaling pathway inhibitor is a molecule blocking PD-1 receptor interactions with PD-1 Ligand 1 (PD-L1) and PD-1 Ligand 2 (PD-L2). In another embodiment, PD-L1 is also known as CD274 or B7-H1. In another embodiment, PD-L2 is also known as CD273 or B7-DC. In another embodiment, the molecule blocking PD-1 receptor interactions with PD-1 Ligand 1 (PD-L1) and PD-1 Ligand 2 (PD-L2) is a molecule interacting with PD-1, PD-L1 or PD-L2. In another embodiment, the molecule blocking PD-1 receptor interactions with PD-1 Ligand 1 (PD-L1) or PD-1 Ligand 2 (PD-L2) is a molecule interacting with PD-1, PD-L1 or PD-L2. The term “interacts” or grammatical equivalents thereof may encompass binding, or coming into contact with another molecule. In another embodiment, the molecule binds to PD-1. In another embodiment, the PD-1 signaling pathway inhibitor is an anti-PD1 antibody.

In one embodiment, a molecule that interacts with PD-1 is a truncated PD-L1 protein. In another embodiment, the truncated PD-L1 protein comprises the cytoplasmic domain of PD-L1 protein. In another embodiment, the molecule interacting with PD-1 is a truncated PD-L2 protein. In another embodiment, the truncated PD-L2 protein comprises the cytoplasmic domain of PD-L2 protein. In another embodiment, the molecule blocking PD-1 receptor interactions with PD-1 Ligand 1 (PD-L1) and PD-1 Ligand 2 (PD-L2) is a molecule interacting with PD-L1 and PD-L2. In another embodiment, the molecule interacting with PD-L1 or PD-L2 is a truncated PD-1 protein, a PD-1 mimic or a small molecule that binds PD-L1 or PD-L2. In another embodiment, the truncated PD-1 protein comprises the cytoplasmic domain of the PD-1 protein.

In one embodiment, an immune checkpoint inhibitor is a CD80/86 signaling pathway inhibitor. CD80 is also known as B7.1 and CD86 is also known as B7.2. It will be appreciated by a skilled artisan that, the CD80/86 signaling pathway inhibitor may encompass an antibody or small molecule that binds to or interacts with CD80/86 and inhibits, suppresses or down-regulates function of the same.

In one embodiment, the immune checkpoint inhibitor is a CTLA-4 signaling pathway inhibitor. CTLA-4 is also known as CD152. It will be appreciated by a skilled artisan that, the CTLA-4 signaling pathway inhibitor may encompass an antibody or small molecule that binds to or interacts with CTLA-4 and inhibits, suppresses or down-regulates function of the same.

In some embodiments, a live-attenuated Listeria strain provided herein is administered before administration of an immunosuppressive molecule antagonist provided herein, while in other embodiments, one of the live-attenuated Listeria strains provided herein is administered after administration of the immunosuppressive molecule antagonist.

Various modes of sequential administration are contemplated by this invention. In one embodiment, an administration regimen comprises administering an immunosuppressive molecule antagonist provided herein followed by administration of a recombinant Listeria vaccine strain provided herein. In another embodiment, the order of administration of components of combination therapy is reversed. In yet another embodiment, administration of one component is immediately followed by administration of the other component. In yet another embodiment, there is an interval between administrations of components. In one embodiment, the interval is at least 1-2 hours. In another embodiment, the interval is at least 2-3 hours. In yet another embodiment, the interval is at least 3-4 hours. In another embodiment, the interval is at least 4-5 hours. In yet another embodiment, the interval is at least 5-6 hours. In another embodiment, the interval is at least 6-8 hours. In yet another embodiment, the interval at least is 8-10 hours. In another embodiment, the interval is at least 10-12 hours. In another embodiment, the interval is at least one day. In another embodiment, the interval is at least two days. In another embodiment, the interval is at least three days. In another embodiment, the interval is at least four days. In another embodiment, the interval is at least five days. In another embodiment, the interval is at least six days. In another embodiment, the interval is at least seven days. In yet another embodiment, the interval is more than seven days.

In some embodiments, at least one of the therapeutic agents in a combination therapy provided herein is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

In one embodiment, the methods provided herein comprise the step of co-administering a composition comprising a recombinant Listeria with an additional therapy. In another embodiment, the additional therapy is surgery, chemotherapy, an immunotherapy, a radiation therapy, an antibody based immunotherapy, or a combination thereof. In another embodiment, the additional therapy precedes administration of a composition comprising a recombinant Listeria. In another embodiment, the additional therapy is administered concurrently with an administration of a composition comprising a recombinant Listeria. In another embodiment, the additional therapy follows administration of the composition comprising a recombinant Listeria. In another embodiment, a composition comprising a recombinant Listeria is administered in increasing doses in order to increase the T-effector cell to regulatory T cell ration and generate a more potent anti-tumor immune response. It will be appreciated by a skilled artisan that the anti-tumor immune response can be further strengthened by providing the subject having a tumor with cytokines including, but not limited to IFN-γ TNF-α, and other cytokines known in the art to enhance cellular immune response, some of which can be found in U.S. Pat. Ser. No. 6,991,785, incorporated by reference herein.

In some embodiments, a composition provided herein is administered to a patient who has not been previously treated with a biotherapeutic or chemotherapeutic agent, i.e., is treatment-naïve. In other embodiments, the composition provided herein is administered to a patient who failed to achieve a sustained response after prior therapy with a biotherapeutic or chemotherapeutic agent, i.e., is treatment-experienced.

It will be appreciated by a skilled artisan that the term “RECIST 1.1 Response Criteria” may encompass the definitions set forth in Eisenhauer et al., E.A. et al., Eur. J Cancer 45:228-247 (2009) for target lesions or non-target lesions, as appropriate based on the context in which response is being measured.

It will be appreciated by a skilled artisan that the tetra “Sustained response” may encompass a sustained therapeutic effect after cessation of treatment with a therapeutic agent, or a composition provided herein. In some embodiments, the sustained response has a duration that is at least the same as the treatment duration, or at least 1.5, 2.0, 2.5 or 3 times longer than the treatment duration.

A composition or combination therapy provided herein is typically used to treat a tumor that is large enough to be found by palpation or by imaging techniques well known in the art, such as MRI, ultrasound, or CAT scan. In some embodiments, a composition or combination therapy provided herein is used to treat an advanced stage tumor having dimensions of at least about 200 mm³·300 mm³, 400 mm³, 500 mm³, 750 mm³, or up to 1000 mm³.

In one embodiment, a combination therapy of the invention is administered to a patient diagnosed with cancer that tests positive for expression of an immunosuppressive molecule such as PD-L1. It will be appreciated by a skilled artisan that expression of an immunosuppressive molecule may be detected using a diagnostic anti-immunosuppressive antibody, or antigen binding fragment thereof, in an IHC assay on an FFPE or frozen tissue section of a tumor sample removed from the patient. Typically, the patient's physician would order a diagnostic test to determine expression of an immunosuppressive molecule in a tumor tissue sample removed from the patient prior to initiation of treatment with a composition comprising an immunosuppressive antagonist and a composition comprising a live-attenuated Listeria strains provided herein, but it is envisioned that the physician could order the first or subsequent diagnostic tests at any time after initiation of treatment, such as for example after completion of a treatment cycle.

In some embodiments, selecting a dosage regimen (also referred to herein as an administration regimen) for composition or combination therapy provided herein depends on several factors, including the serum or tissue turnover rate of the entity, the level of symptoms, the immunogenicity of the entity, and the accessibility of the target cells, tissue or organ in the individual being treated. Preferably, a dosage regimen maximizes the amount of each therapeutic agent delivered to the patient consistent with an acceptable level of side effects. Accordingly, the dose amount and dosing frequency of a composition provided herein or each therapeutic agent (or active ingredient) in a combination therapy provided herein depends in part on the particular therapeutic agent, the severity of the cancer being treated, and patient characteristics. Guidance in selecting appropriate doses of antibodies, cytokines, and small molecules, which may be used as an additional therapy are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). It will be appreciated by a skilled artisan that deteunination of an appropriate dosage regimen may be made by the clinician, e.g., using parameters or factors known or suspected in the art to affect treatment or predicted to affect treatment, and will depend, for example, the patient's clinical history (e.g., previous therapy), the type and stage of the disease or cancer to be treated and biomarkers of response to one or more of the therapeutic agents in a composition or combination therapy provided herein.

Biotherapeutic agents in a combination therapy of the invention may be administered by continuous infusion, or by doses at intervals of, e.g., daily, every other day, three times per week, or one time each week, two weeks, three weeks, monthly, bimonthly, etc. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144.

In some embodiments that employ an anti-human PD-1 mAb as the PD-1 immunosuppressive antagonist in the combination therapy, the dosing regimen will comprise administering the anti-human PD-1 mAb at a flat dose of 100 to 500 mg or a weight-based dose of 1 to 10 mg/kg at intervals of about 14 days (±2 days) or about 21 days (±2 days) or about 30 days (+2 days) throughout the course of treatment.

In other embodiments that employ an anti-human PD-1 mAb as the PD-1 antagonist in the combination therapy, the dosing regimen will comprise administering the anti-human PD-1 mAb at a dose of from about 0.005 mg/kg to about 10 mg/kg, with intra-patient dose escalation. In other escalating dose embodiments, the interval between doses will be progressively shortened, e.g., about 30 days (±2 days) between the first and second dose, about 14 days (±2 days) between the second and third doses. In certain embodiments, the dosing interval will be about 14 days (±2 days), for doses subsequent to the second dose.

In one embodiment, the terms “treatment regimen”, “dosing protocol” and “dosing regimen” are used interchangeably herein and encompass the dose and timing of administration of each therapeutic agent in a combination of the invention.

In certain embodiments, a subject will be administered an intravenous (IV) infusion of a composition comprising any of the immunosuppressive molecules antagonists described herein.

In one embodiment, of the invention, the PD-1 antagonist in the combination therapy is nivolumab, which is administered intravenously at a dose selected from the group consisting of: 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg Q3W.

In another embodiment, of the invention, the PD-1 antagonist in the combination therapy PD-1 antagonist is administered in a liquid medicament at a dose selected from the group consisting of 200 mg Q3W, 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg Q3W or equivalents of any of these doses (e.g., a PK model of a PD-1 antagonist estimates that the fixed dose of 200 mg Q3W provides exposures that are consistent with those obtained with 2 mg/kg Q3W). In some embodiments, a PD-1 antagonist is administered as a liquid medicament which comprises 25 mg/ml the PD-1 antagonist, 7% (w/v) sucrose, 0.02% (w/v) polysorbate 80 in 10 mM histidine buffer pH 5.5, and the selected dose of the medicament is administered by IV infusion over a time period of 30 minutes +/−10 min.

In some embodiments, pharmaceutical compositions containing strains of the present invention and compositions comprising an immunosuppressive antagonist are administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally, intra-tumorally or via the enteral route. It will be appreciated by a skilled artisan that the term “enteral route” of administration may encompass the administration via any part of the gastrointestinal tract. Examples of enteral routes include oral, mucosal, buccal, and rectal route, or intragastric route. It will also be appreciated by a skilled artisan that the term “Parenteral route” of administration may encompass a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration.

In addition, the antibodies and compositions provided herein can be administered using any suitable method, such as by oral ingestion, nasogastric tube, gastrostomy tube, injection, infusion, implantable infusion pump, and osmotic pump. The suitable route and method of administration may vary depending on a number of factors such as the specific antibody being used, the rate of absorption desired, specific formulation or dosage form used, type or severity of the disorder being treated, the specific site of action, and conditions of the patient, and can be readily selected by a person skilled in the art.

It will be appreciated by a skilled artisan that when a compositions provided herein are administered orally, these compositions are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment, the active ingredient is formulated in a capsule. In accordance with this embodiment, the compositions of the present invention comprise, in addition to the active compound and the inert carrier or diluent, a hard gelating capsule.

In another embodiment, a composition comprising a recombinant Listeria strain is administered by intravenous, intra-arterial, or intra-muscular injection of a liquid preparation. Suitable liquid formulations include solutions, suspensions, dispersions, emulsions, oils and the like. In one embodiment, pharmaceutical compositions comprising a recombinant Listeria strain are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In one embodiment, a vaccine of the methods and compositions provided herein may be administered to a host vertebrate animal, preferably a mammal, and more preferably a human, either alone or in combination with a pharmaceutically acceptable carrier. In another embodiment, a vaccine is administered in an amount effective to induce an immune response to the Listeria strain itself or to a heterologous antigen which the Listeria species has been modified to express. In another embodiment, the amount of vaccine or immunogenic composition to be administered may be routinely determined by one of skill in the art when in possession of the present disclosure. In another embodiment, a pharmaceutically acceptable carrier may include, but is not limited to, sterile distilled water, saline, phosphate buffered solutions or bicarbonate buffered solutions. In another embodiment, the pharmaceutically acceptable carrier selected and the amount of carrier to be used will depend upon several factors including the mode of administration, the strain of Listeria and the age and disease state of the vaccinee. In another embodiment, administration of the vaccine may be by an oral route, or it may be parenteral, intranasal, intramuscular, intravascular, intrarectal, intraperitoneal, or any one of a variety of well-known routes of administration. In another embodiment, the route of administration may be selected in accordance with the type of infectious agent or tumor to be treated.

In another embodiment, the present invention provides a method of treating, suppressing, or inhibiting at least one tumor in a subject comprising administering an immunogenic composition provided herein.

In some embodiments an attenuated bacteria, or attenuated Listeria, is administered as a liquid medicament, and the selected dose of the medicament is administered by IV infusion over a time period of 30 minutes +/−10 min.

The optimal dose for a combination therapy comprising an immunosuppressive antagonist provided herein in combination with a live-attenuated Listeria strain provided herein is identified by dose escalation of one or both of these agents. In another embodiment, the optimal dose for a composition comprising either the anti-immunosuppressive antagonist provided herein or the live-attenuated Listeria strain provided herein is identified by dose escalation of one or both of these agents.

In one embodiment, a patient is treated with the combination therapy provided herein on day 1 of weeks 1, 4 and 7 in a 12 week cycle, starting with an immunosuppressive antagonist being administered at a starting dose of 50, 100, 150, or 200 mg, and a live-attenuated Listeria strain provided herein at a starting dose of ranging from about 1×10⁷ CFU to about 5.0×10¹⁰ CFU.

In an embodiment, a composition comprising an immunosuppressive antagonist infusion is administered first, followed by a NSAIDS, e.g., naproxen or ibuprofen, and oral antiemetic medication within a predetermined amount of time prior to administration of a live-attenuated Listeria strain provided herein. In another embodiment, the predetermined amount of time is 5-10 min, 11-20 min, 21-40 min, 41-60 min. In another embodiment, the predetermined amount of time is at least one hour. In another embodiment, the predetermined amount of time is 1-2 hours, 2-4 hours, 4-6 hours, 6-10 hours. In another embodiment, administrations of a NSAIDS, e.g., naproxen or ibuprofen, and oral antiemetic medication is repeated on a need basis to the subject, prior to administration of a live-attenuated Listeria strain provided herein.

In another embodiment, a composition comprising an immunosuppressive antagonist is administered at a starting dose of 50, 100, 150 or 200 mg Q3W and a live-attenuated Listeria strain provided herein is administered Q3W at a starting dose of between 1×10⁷ and 3.5×10¹⁰ CFU.

In another embodiment, a composition comprising a live-attenuated Listeria strain provided herein is administered at a starting dose of 5×10⁹ Q3W and an anti-PD-1 antibody is administered at a starting dose of 200 mg Q3W, and if the starting dose of the combination is not tolerated by the patient, then the dose of the live-attenuated Listeria strain provided herein is reduced to 1×10⁹ cfu Q3W or the dose of the anti-PD-1 antibody is reduced to 150 mg Q3W. It is to be understood by a skilled artisan that the doses of any of the components of a combination therapy provided herein may be incrementally adjusted to a lower or higher dose, as further provided herein, based on a subject's response to the combination therapy.

In some embodiments, dosage levels below the lower limit of the aforesaid range may be more than adequate, while in other cases still larger doses may be employed, as determined by those skilled in the art.

In some embodiments, a treatment cycle begins with the first day of combination treatment and lasts for at least 12 weeks, 24 weeks or 48 weeks. On any day of a treatment cycle that the drugs are co-administered, the timing between the separate IV infusions of an immunosuppressive antagonist and a live-attenuated Listeria strain provided herein is between about 15 minutes to about 45 minutes. The invention contemplates that an immunosuppressive antagonist and a live-attenuated Listeria strain provided herein may be administered in either order or by simultaneous IV infusion.

In some embodiments, the combination therapy is administered for at least 2 to 4 weeks after the patient achieves a CR.

In some embodiments, a patient selected for treatment with the combination therapy of the invention has been diagnosed with a metastatic cancer and the patient has progressed or become resistant to no more than 2 prior systemic treatment regimens. In some embodiments, a patient selected for treatment with the combination therapy of the invention has been diagnosed with a metastatic cancer and the patient has progressed or become resistant to no more than 3 prior systemic treatment regimens.

In one embodiment, an immunosuppressive antagonist may be produced in a producing cell line known in the art, such as, but not limited to CHO cells using conventional cell culture and recovery/purification technologies.

In some embodiments, a medicament comprising an immunosuppressive antagonist provided herein may be provided as a liquid formulation or prepared by reconstituting a lyophilized powder with sterile water for injection prior to use. WO 2012/135408 describes the preparation of liquid and lyophilized medicaments comprising an anti-PD-1 antibody that are suitable for use in the present invention. In some embodiments, a medicament comprising an anti-PD-1 antibody is provided in a glass vial which contains about 50 mg of anti-PD-1 antibody.

The present invention also provides a medicament which comprises a live-attenuated Listeria strain provided herein and a pharmaceutically acceptable excipient.

An immunosuppressive antagonist medicament and/or a live-attenuated Listeria strain medicament provided herein may be provided as a kit which comprises a first container and a second container and a package insert. The first container contains at least one dose of a medicament comprising an immunosuppressive antagonist, the second container contains at least one dose of a medicament comprising a live-attenuated Listeria strain provided herein, and the package insert, or label, which comprises instructions for treating a patient for a cancer using the medicaments. The first and second containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. In some embodiments of the kit, the immunosuppressive antagonist is an anti-PD-1 antibody and the instructions state that the medicaments are intended for use in treating a patient having a PD-L1 expressing cancer that tests positive for PD-L1 expression by an IHC assay.

It will be appreciated by a skilled artisan that the term “comprise” or grammatical forms thereof, may encompass the inclusion of the indicated active agent, such as the Lm strains of this invention, as well as inclusion of other active agents, such as an antibody or functional fragment thereof, and pharmaceutically acceptable carriers, excipients, emollients, stabilizers, etc., as are known in the pharmaceutical industry. In some embodiments, the term “consisting essentially of” may encompass a composition, whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may encompass components, which exert a therapeutic effect via a mechanism distinct from that of the indicated active ingredient. In some embodiments, the term “consisting essentially of” may encompass components, which exert a therapeutic effect and belong to a class of compounds distinct from that of the indicated active ingredient. In some embodiments, the term “consisting essentially of” may encompass components, which exert a therapeutic effect and may be distinct from that of the indicated active ingredient, by acting via a different mechanism of action. In some embodiments, the term “consisting essentially of” may encompass components which facilitate the release of the active ingredient. In some embodiments, the term “consisting” may encompass a composition, which contains the active ingredient and a pharmaceutically acceptable carrier or excipient.

It is understood that wherever embodiments are described herein with the language “comprising”, otherwise analogous embodiments described in terms of “consisting of” and/or “consisting essentially of” are also provided.

In one embodiment, the singular forms of words such as “a,” “an,” and “the,” include their corresponding plural references unless the context clearly dictates otherwise.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

It will be appreciated by a skilled artisan that the term “About” when used to modify a numerically defined parameter (e.g., the dose of a PD-1 antagonist or the length of treatment time with a combination therapy described herein) may encompass variation of the parameter in quantitative terms plus or minus 5%, or in another embodiment plus or minus 10%, or in another embodiment plus or minus 15%, or in another embodiment plus or minus 20% of stated numerical value for that parameter. For example, a dose of about 200 mg of the PD-1 antagonist, may vary between 180 mg and 220 mg.

It is to be understood by the skilled artisan that the term “subject” can encompass a mammal including an adult human or a human child, teenager or adolescent in need of therapy for, or susceptible to, a condition or its sequelae, and also may include non-human mammals such as dogs, cats, pigs, cows, sheep, goats, horses, rats, and mice. It will also be appreciated that the term may encompass livestock. The term “subject” does not exclude an individual that is normal in all respects.

It will be appreciated by the skilled artisan that the term “mammal” for purposes of treatment refers to any animal classified as a mammal, including, but not limited to, humans, domestic and farm animals, and zoo, sports, or pet animals, such as canines, including dogs, and horses, cats, cattle, pigs, sheep, etc.

In the following examples, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Thus these examples should in no way be construed, as limiting the broad scope of the invention.

### EXAMPLES

### Materials & Methods

Construction of Plasmid pAdv142 and Strain LmddA142

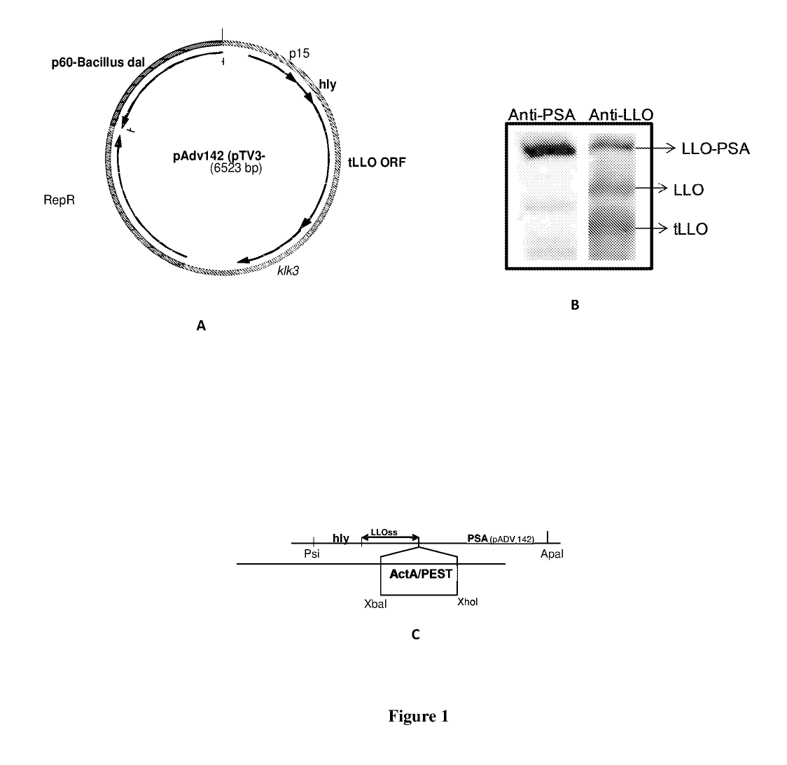

This plasmid is next generation of the antibiotic free plasmid, pTV3 that was previously constructed by Verch et al. The unnecessary copy of the virulence gene transcription activator, prfA was deleted from plasmid pTV3 since Lm-ddA contains a copy of prfA gene in the chromosome. Therefore, the presence of prfA gene in the dal containing plasmid was not essential. Additionally, the cassette for p60-Listeria dal at the Nhel/Pacl restriction site was replaced by p60-Bacillus subtilis dal (dal_{Bs}) resulting in the plasmid pAdv134. Further, pAdv134 was restricted with XhoI/XmaI to clone human PSA, klk3 resulting in the plasmid, pAdv142. The new plasmid pAdv 142 (FIG. 1) contains dal_{Bs} and its expression was under the control of Lm p60 promoter. The shuttle plasmid pAdv142 could complement the growth of both E. coli ala drx MB2159 as well as Lmdd in the absence of exogenous addition of D-alanine. The antigen expression cassette in the plasmid pAdv 142 consists of hly promoter and tLLO-PSA fusion protein (FIG. 1).

The plasmid pAdv142 was transformed to the Listeria background strain, LmddA resulting in LmddA142 or ADXS31-142. The expression and secretion of LLO-PSA fusion protein by the strain, ADXS31-142 was confirmed by western analysis using anti-LLO and anti-PSA antibody and is shown in FIG. 1. There was stable expression and secretion of LLO-PSA fusion protein by the strain, ADXS31-142 after two in vivo passages in C57BL/6 mice.

### Construction of LmddA211, LmddA223 and LmddA224 Strains

The different ActA/PEST regions were cloned in the plasmid pAdv142 to create the three different plasmids pAdv211, pAdv223 and pAdv224 containing different truncated fragments of ActA protein.

LLO Signal Sequence (LLOss)-ActAPEST2 (pAdv211)/LnldA211

First two fragments Psil-LLOss-XbaI (817 bp in size) and LLOss-XbaI-ActA-PEST2 (602 bp in size) were amplified and then fused together by using SOEing PCR method with an overlap of 25 bases. This PCR product now contains PsiI-LLOss-Xbal-ActAPEST2-XhoI a fragment of 762 bp in size. The new Psil-LLOss-Xbal-ActAPEST2-XhoI PCR product and pAdv142 (LmddA-PSA) plasmid were digested with PsiI/XhoI restriction enzymes and purified. Ligation was set up and transformed into MB2159 electro competent cells and plated onto LB agar plates. The Psil-LLOss-Xbal-ActAPEST2/pAdv 142 (PSA) clones were selected and screened by insert-specific PCR reaction PsiI-LLOss-Xbal-ActAPEST2/pAdv 142 (PSA) clones #9, 10 were positive and the plasmid purified by mini preparation. Following screening of the clones by PCR screen, the inserts from positive clones were sequenced. The plasmid Psil-LLOss-Xbal-ActAPEST2/pAdv 142 (PSA) referred as pAdv211.10 was transformed into Listeria LmddA mutant electro competent cells and plated onto BHI/strep agar plates. The resulting LmddA211 strain was screened by colony PCR. Several Listeria colonies were selected and screened for the expression and secretion of endogenous LLO and ActAPEST2-PSA (LA229-PSA) proteins. There was stable expression of ActAPEST2-PSA fusion proteins after two in vivo passages in mice.

### LLOss-ActAPEST3 and PEST4:

ActAPEST3 and ActAPEST4 fragments were created by PCR method. PCR products containing LLOss-XbaI-ActAPEST3-XhoI (839 bp in size) and LLOss-XbaI-ActAPEST4-XhoI a fragments (1146 bp in size) were cloned in pAdv142. The resulting plasmid pAdv223 (Psil-LLOss-Xbal-ActAPEST3-XhoI/pAdv 142) and pAdv224 (Psil-LLOss-Xbal-ActAPEST4/pAdv 142) clones were selected and screened by insert-specific PCR reaction. The plasmids pAdv223 and pAdv224 were transformed to the LmddA backbone resulting in LmddA223 and LmddA224, respectively. Several Listeria colonies were selected and screened for the expression and secretion of endogenous LLO, ActAPEST3-PSA (LmddA223) or ActAPEST4-PSA (LmddA224) proteins. There was stable expression and secretion of the fusion protein ActAPEST3-PSA (LmddA223) or ActAPEST4-PSA (LmddA224) after two in vivo passages in mice.

Experimental plan 1

The therapeutic efficacy of the ActA-PEST-PSA (PEST3, PEST2 and PEST4 sequences) and tLLO-PSA using TPSA23 (PSA expressing tumor model) were evaluated and compared. Untreated mice were used as control group. In parallel evaluated the immune responses were also using intracellular cytokine staining for interferon-gamma and PSA tetramer staining.

For the tumor regression study. Ten groups of eight C57BL/6 mice (7 weeks old males) were implanted subcutaneously with 1×10⁶ of TPSA23 cells on day 0. On Day 6 they received immunization which was followed by 2 booster doses which were 1 week apart. Tumor growth was monitored every week until they reached a size of 1.2 cm in average diameter.

### Immunogenicity Study.

2 groups of C57BL/6 mice (7 weeks old males) were immunized 3 times with one week interval with the vaccines listed in the table below. Six days after the last boost injection, mice were sacrificed, and the spleens will be harvested and the immune responses were tested for tetramer staining and IFN-γ secretion by intracellular cytokine staining.

### Experimental Plan 2

This experiment was a repeat of Experimental plan 1, however, the Naïve, tLLO, ActA/PEST2-PSA and tLLO-PSA groups were only included. Similar to Experimental plan 1, the therapeutic efficacy was evaluated using TPSA23 (PSA expressing tumor model). Five C57BL/6 mice per group were implanted subcutaneously with 1×10⁶ of TPSA23 cells on day 0. On Day 6 they received immunization (1×10⁸ CFU/mL) which was followed by booster 1 week later. Spleen and tumor was collected on day 6 post last treatment. The immune response was monitored using PSA pentamer staining in both spleen and tumor.

### Materials & Methods:

TPSA23 cells are cultured in complete medium. Two days prior to implanting tumor cells in mice, TPSA23 cells were sub-cultured in complete media. On the day of the experiment (Day 0), cells were trypsinized and washed twice with PBS. Cells were counted and re-suspended at a concentration of 1×10⁶ cells/200 ul in PBS/mouse for injection. Tumor cells were injected subcutaneously in the flank of each mouse.

### Complete Medium for TPSA23 Cells

Complete medium for TPSA23 cells was prepared by mixing 430 ml of DMEM with Glucose, 45 ml of fetal calf serum (FCS), 25 ml of Nu-Serum IV, 5 ml 100× L-Glutamine, 5 ml of 100 mM Na-Pyruvate, 5 ml of 10,000 U/mL Penicillin/Streptomycin. 0.005 mg/ml of Bovine Insulin and 10 nM of Dehydroisoandrosterone was added to the flask while splitting cells.

Complete Medium for Splenocytes (c-RPMI)

Complete medium was prepared by mixing 450 ml of RPMI 1640, 50 ml of fetal calf serum (FCS), 5 ml of 1M HEPES, 5 ml of 100× Non-essential amino acids (NEAA), 5 ml of 100× L-Glutamine, 5 ml of 100 mM Na-Pyruvate, 5 ml of 10,000 U/mL Penicillin/Streptomycin and 129 ul of 14.6M 2-Mercaptoethanol.

### Preparing Isolated Splenocytes

Work was performed in biohazard hood. Spleens were harvested from experimental and control mice groups using sterile forceps and scissors. They were transport in 15 ml tubes containing 10 ml PBS to the lab. Spleen from each mouse was processed separately. Spleen was taken in a sterile Petri dish and mashed using the back of plunger from a 3 mL syringe. Spleen cells were transferred to a 15 ml tube containing 10 ml of RPMI 1640. Cells were pelleted by centrifugation at 1,000 RPM for 5 min at 4° C. The supernatant was discarded in 10% bleach. Cell pellet was gently broken by tapping. RBC was lysed by adding 2 ml of RBC lysis buffer per spleen to the cell pellet. RBC lysis was allowed for 2 min. Immediately, 10 ml of c-RPMI medium was added to the cell suspension to deactivate RBC lysis buffer. Cells were pelleted by centrifugation at 1,000 RPM for 5 min at 4° C. The supernatant was discarded and cell pellet was re-suspended in 10 ml of c-RPMI and passed through a cell strainer. Cells were counted using hemocytometer and the viability was checked by mixing 10 ul of cell suspension with 90 ul of Trypan blue stain. About 2×10⁶ cells were used for pentamer staining. (Note: each spleen should yield 1-2×10⁸ cells).

Preparing Single Cell Suspension from Tumors Using Miltenyi Mouse Tumor Dissociation Kit

Enzyme mix was prepared by adding 2.35 mL of RPMI 1640, 100 μL of Enzyme D, 50 uL of Enzyme R, and 12.5 μL of Enzyme A into a gentleMACS C Tube. Tumor (0.04-1 g) was cut into small pieces of 2-4 mm and transferred into the gentleMACS C Tube containing the enzyme mix. The tube was attached upside down onto the sleeve of the gentleMACS Dissociator and the Program m_impTumor_02 was run. After termination of the program, C Tube was detached from the gentleMACS Dissociator. The sample was incubated for 40 minutes at 37° C. with continuous rotation using the MACSmix Tube Rotator. After completion of incubation the C tube was again attached upside down onto the sleeve of the gentleMACS Dissociator and the program m_impTumor_03 was run twice. The cell suspension was filtered through 70 μm filter placed on a 15 mL tube. The filter was also washed with 10 mL of RPMI 1640. The cells were centrifuged at 300×g for 7 minutes. The supernatant was discarded and the cells were re-suspended in 10 ml of RPMI 1640. At this point one can divide the cells for pentamer staining.

### Pentamer Staining of Splenocytes

The PSA-specific T cells were detected using commercially available PSA-H-2D^{b} pentamer from Prolmmune using manufacturers recommended protocol. Splenocytes were stained for CD8, CD62L, CD3 and Pentamer. While tumor cells were stained for CD8, CD62L, CD45 and Pentamer. The CD3⁻CD8⁺ CD62L^{low} cells were gated to determine the frequency of CD3⁺CD8⁺CD62L^{low} PSA pentamer⁺cells. The stained cells were acquired and analyzed on FACS Calibur using Cell quest software.

### Materials Needed for Pentamer Staining

Splenocyptes (preparation described above), Pro5® Recombinant MHC PSA Pentamer conjugated to PE. (Note: Ensure that the stock Pentamer is stored consistently at 4° C. in the dark, with the lid tightly closed), anti-CD3 antibody conjugated to PerCP Cy5.5, anti-CD8 antibody conjugated to FITC and anti-CD62L antibody conjugated to APC, wash buffer (0.1% BSA in PBS) and fix solution (1% heat inactivated fetal calf serum (HI-FCBS), 2.5% formaldehyde in PBS)

### Standard Staining Protocol

Pro50 PSA Pentamer was centrifuged in a chilled microcentrifuge at 14,000×g for 5-10 minutes to remove any protein aggregates present in the solution. These aggregates may contribute to non-specific staining if included in test volume. 2×10⁶ splenocytes were allocated per staining condition and 1 ml of wash buffer was added per tube. Cells were centrifuged at 500×g for 5 min in a chilled centrifuge at 4° C. The cell pellet was re-suspended in the residual volume (˜50 μl ). All tubes were chilled on ice for all subsequent steps, except where otherwise indicated. 10 μl of labeled Pentamer was added to the cells and mixed by pipetting. The cells were incubated at room temperature (22° C.) for 10 minutes, shielded from light. Cells were washed with 2 ml of wash buffer per tube and re-suspend in residual liquid (˜50 μl ). An optimal amount of anti-CD3, anti-CD8 and anti-CD62L antibodies were added (1:100 dilution) and mixed by pipetting. Single stain control samples were also made at this point. Samples were incubated on ice for 20 minutes, shielded from light. Cells were washed twice with 2 ml wash buffer per tube. The cell pellet was re-suspended in the residual volume (˜50 μl ). 200 μl of fix solution was added to each tube and vortexed. The tubes were stored in dark in the refrigerator until ready for data acquisition. (Note: the morphology of the cell changes after fixing, so it is advisable to leave the samples for 3 hours before proceeding with data acquisition. Samples can be stored for up to 2 days).

Intracellular Cytokine Staining (IFN-γ) protocol:

2×10⁷ cells/ml splenocytes were taken in FACS tubes and 100 μl of Brefeldin A (BD Golgi Plug) was added to the tube. For stimulation, 2 μl M Peptide was added to the tube and the cells were incubated at room temperature for 10-15 minutes. For positive control samples, PMA (10 ng/ml) (2×) and ionomycin (1 μl g/ml) (2×) was added to corresponding tubes. 100 μl of medium from each treatment was added to the corresponding wells in a U-bottom 96-well plate. 100 μl of cells were added to the corresponding wells (200 μl final volume—medium+cells). The plate was centrifuged at 600 rpm for 2 minutes and incubated at 37° C. 5% CO₂ for 5 hours. Contents from the plate was transferred to FACS tubes. 1 ml of FACS buffer was added to each tube and centrifuged at 1200 rpm for 5 min. The supernatant was discarded. 200 μl of 2.4 G2 supernatant and 10 μl of rabbit serum was added to the cells and incubated for 10 minutes at room temperature. The cells were washed with 1 mL of FACS buffer. The cells were collected by centrifugation at 1200 rpm for 5 minutes. Cells were suspended in 50 μl of FACS buffer containing the fluorochrome-conjugated monoclonal antibodies (CD8 FITC, CD3 PerCP-Cy5.5, CD62L APC) and incubated at 4° C. for 30 minutes in the dark. Cells were washed twice with 1 mL FACS buffer and re-suspended in 200 μl of 4% formalin solution and incubated at 4° C. for 20 min. The cells were washed twice with 1 mL FACS buffer and re-suspended in BD Perm/Wash (0.25 ml/tube) for 15 minutes. Cells were collected by centrifugation and re-suspended in 50 μl of BD Peim/Wash solution containing the fluorochrome-conjugated monoclonal antibody for the cytokine of interest (IFNg-PE). The cells were incubated at 4° C. for 30 minutes in the dark. Cells were washed twice using BD Perm/Wash (1 ml per tube) and re-suspended in 200 μl FACS buffer prior to analysis.

### Results

### Example 1

### Vaccination with Recombinant Listeria Constructs Leads to Tumor Regression

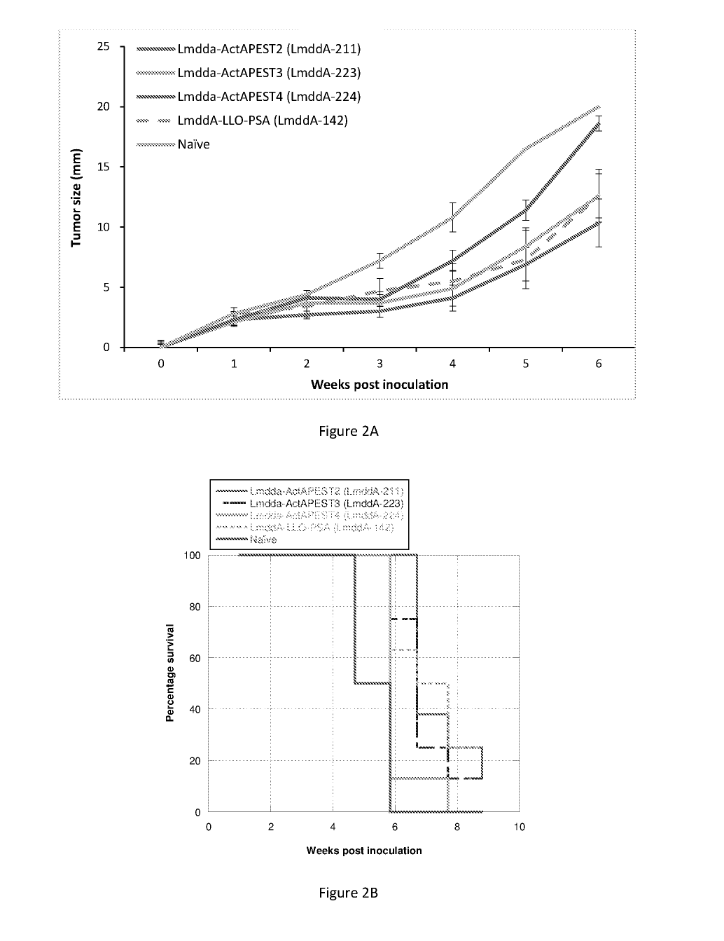

The data showed that by week 1, all groups had developed tumor with the average size of 2-3 mm. On week 3 (Day 20) mice immunized with ActAPEST (2, 3 and 4)-PSA and LmddA-142 (ADXS31-142), which expresses a tLLO fused to PSA showed, tumor regression and slow down of the tumor growth. By week 6, all mice in naïve and most in ActAPEST4-PSA treated group had big tumors and had to be euthanized (FIG. 2). However, LmddA-142, ActA-PEST2 and ActA-PEST3 mice groups showed better tumor regression and survival rate (FIG. 2).

### Example 2

### Vaccination with Recombinant Listeria Generates High Levels of Antigen-specific T Cells

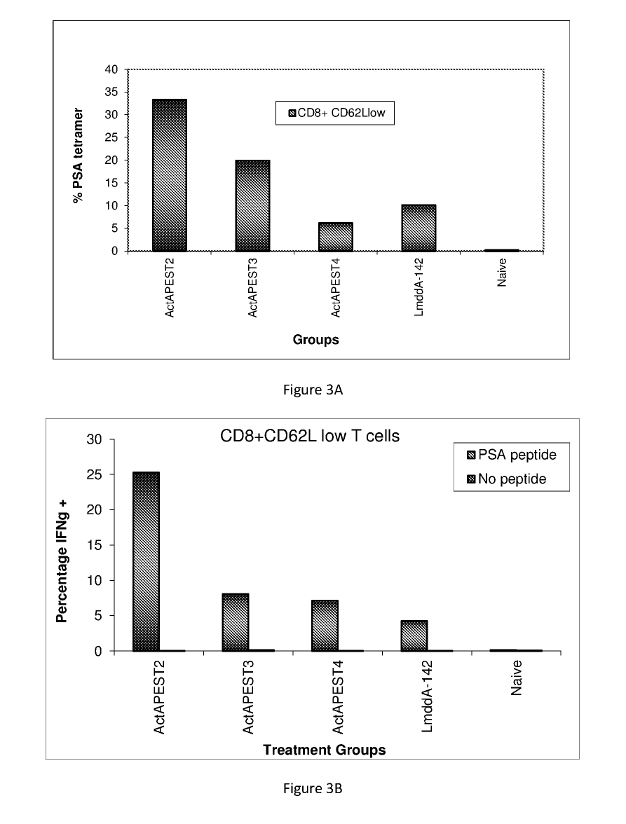

LmddA-ActAPEST2-PSA vaccine generated high levels of PSA-specific T cells response compared to LmddA-ActAPEST (3 or 4)-PSA, or LmddA-142 (FIG. 3). The magnitude of PSA tetramer specific T cells in PSA-specific vaccines was 30 fold higher than naive mice. Similarly, higher levels of IFN-γ secretion was observed for LmddA-ActAPEST2-PSA vaccine in response to stimulation with PSA-specific antigen (FIG. 3).

### Example 3

### Vaccination with ACTA/PEST2 (LA229) Generates A High Number of Antigen-Specific CD8+ T Cells In Spleen

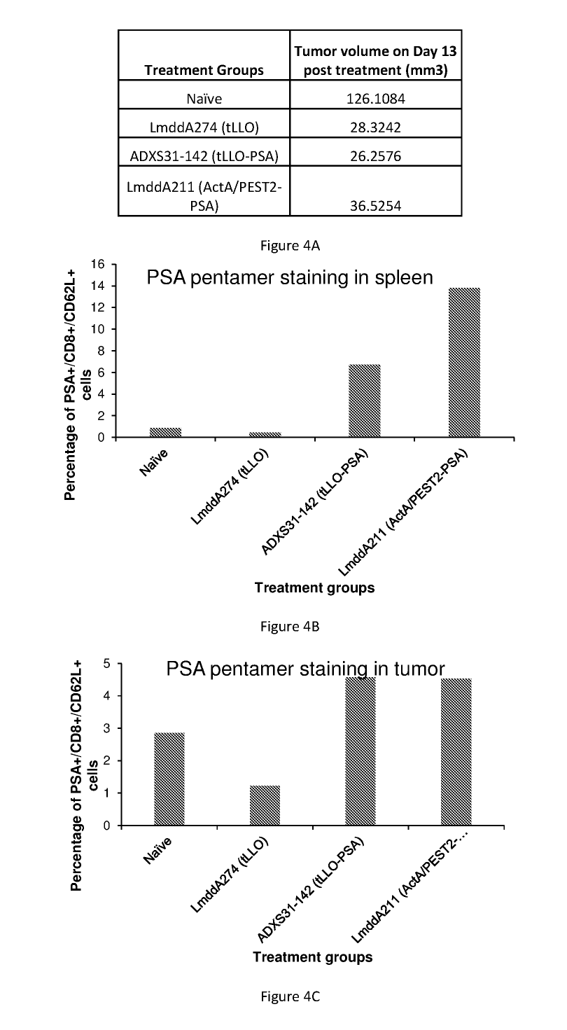

Lm expressing ActA/PEST2 fused PSA was able to generate higher numbers of PSA specific CD8+ T cells in spleen compared to Lm expressing tLLO fused PSA or tLLO treated group. The number of PSA specific CD8+ T cells infiltrating tumors were similar for both Lm-tLLO-PSA and Lm-ActA/PEST2-PSA immunized mice (FIG. 4). Also, tumor regression ability of Lm expressing ActA/PEST2-PSA was similar to that seen for LmddA-142 which expresses tLLO-PSA (FIG. 4).

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A recombinant Listeria strain comprising a nucleic acid molecule comprising a first open reading frame encoding a recombinant polypeptide, said polypeptide comprising a truncated ActA protein fused to an antigen.

2. The recombinant Listeria of claim 1, wherein said antigen is a heterologous antigen or a self-antigen.

3. A recombinant Listeria strain comprising a nucleic acid molecule comprising a first open reading frame encoding a recombinant polypeptide, said polypeptide comprising a truncated ActA protein.

4. The recombinant Listeria strain of any one of claims 1-3, wherein said truncated ActA protein is selected from the sequences set forth in SEQ ID NO: 9-14.

5. The recombinant Listeria strain of any one of claims 1-4, wherein said recombinant Listeria is Listeria monocytogenes.

6. The recombinant Listeria strain of any one of claims 1-5, wherein said Listeria comprises a genomic mutation in the D-amino acid transferase gene, and the D-alanine racemase gene.

7. The recombinant Listeria strain of any one of claims 1-6, wherein said nucleic acid molecule further comprises a second open reading frame encoding a second open reading frame encoding a metabolic enzyme, and wherein said metabolic enzyme complements an endogenous gene that is mutated, deleted or inactivated in the chromosome of said recombinant Listeria strain.

8. The recombinant Listeria strain of any one of claims 1-7, wherein said metabolic enzyme encoded by said second open reading frame is an amino acid metabolism enzyme.

9. The recombinant Listeria strain of any one of claims 1-8, wherein said metabolic enzyme encoded by said second open reading frame is an alanine racemase enzyme or a D-amino acid transferase enzyme.

10. The recombinant Listeria strain of any one of claims 1-9, wherein said nucleic acid molecule in said recombinant Listeria further comprises a third open reading frame encoding a metabolic enzyme.

11. The recombinant Listeria strain of claim 10, wherein said metabolic enzyme encoded by said third open reading frame is a D-amino acid transferase enzyme or an alanine racemase enzyme.

12. The recombinant Listeria strain of any one of claims 1-11, wherein said nucleic acid molecule is integrated into the Listeria genome.

13. The recombinant Listeria strain of any one of claims 1-12, wherein said nucleic acid molecule is in a plasmid in said recombinant Listeria strain.

14. The recombinant Listeria strain of claim 13, wherein said plasmid is stably maintained in said recombinant Listeria strain in the absence of antibiotic selection.

15. The recombinant Listeria strain of claim 14, wherein said plasmid does not confer antibiotic resistance upon said recombinant Listeria.

16. The recombinant Listeria strain of any one of claims 1-15, wherein said recombinant Listeria strain is attenuated.

17. The recombinant Listeria strain of any one of claims 1-16, wherein said recombinant Listeria comprises a mutation in the ActA virulence gene.

18. The recombinant Listeria strain of any one of claims 1-17, wherein said recombinant Listeria strain has been passaged through an animal host.

19. The recombinant Listeria strain of any one of claims 1-18, further comprising an adjuvant.

20. The recombinant Listeria strain of claim 19, wherein said adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

21. The recombinant Listeria strain of any one of claims 1 and 4-20, wherein said heterologous antigen is an infectious disease antigen, a parasitic antigen, or a tumor antigen.

22. A recombinant polypeptide comprising the truncated ActA protein of claim 4.

23. The recombinant polypeptide of claim 22, further comprising a heterologous antigen.

24. A recombinant nucleic acid encoding the recombinant polypeptide of any one of claims 21-22.

25. A pharmaceutical composition comprising the recombinant Listeria strain of any one of claims 1-21, the recombinant polypeptide of any one of claims 22-23, or the recombinant nucleic acid of claim 24, and a pharmaceutically acceptable carrier.

26. A method of inducing an anti-disease immune response in a subject, the method comprising the step of administering a composition comprising a recombinant Listeria strain, said Listeria strain comprising a recombinant nucleic acid comprising a first open reading frame encoding a recombinant polypeptide, said recombinant polypeptide comprising a truncated ActA fused to an antigen.

27. The method of claim 26, wherein said antigen is a heterologous antigen or a self-antigen.

28. The method of any one of claims 26-27, wherein said truncated ActA protein is selected from the sequences set forth in SEQ ID NO: 9-14.

29. The method of any one of claims 27-28, wherein said recombinant Listeria is Listeria monocytogenes.

30. The method of claim 29, wherein said Listeria comprises a genomic mutation in the D-amino acid transferase gene, and the D-alanine racemase gene.

31. The method of any one of claims 26-30, wherein said nucleic acid molecule further comprises a second open reading frame encoding a second open reading frame encoding a metabolic enzyme, and wherein said metabolic enzyme complements an endogenous gene that is mutated in the chromosome of said recombinant Listeria strain.

32. The method of any one of claims 26-31, wherein said metabolic enzyme encoded by said second open reading frame is an amino acid metabolism enzyme.

33. The method of any one of claims 26-32, wherein said metabolic enzyme encoded by said second open reading frame is an alanine racemase enzyme or a D-amino acid transferase enzyme.

34. The method of any one of claims 26-33, wherein said nucleic acid molecule in said recombinant Listeria further comprises a third open reading frame.

35. The method of claim 34, wherein said metabolic enzyme encoded by said third open reading frame is a D-amino acid transferase enzyme or an alanine racemase enzyme.

36. The method of any one of claims 26-35, wherein said nucleic acid molecule is integrated into the Listeria genome.

37. The method of any one of claims 26-36, wherein said nucleic acid molecule is in a plasmid in said recombinant Listeria vaccine strain.

38. The method of claim 37, wherein said plasmid is stably maintained in said recombinant Listeria vaccine strain in the absence of antibiotic selection.

39. The method of claim 38, wherein said plasmid does not confer antibiotic resistance upon said recombinant Listeria.

40. The method of any one of claims 26-39, wherein said recombinant Listeria strain is attenuated.

41. The method of any one of claims 26-39, wherein said recombinant Listeria comprises a mutation in the ActA virulence gene.

42. The method of any one of claims 26-39, wherein said recombinant Listeria strain has been passaged through an animal host.

43. The method of any one of claims 26-42, further comprising the step of administering an adjuvant.

44. The method of claim 43, wherein said adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

45. The method of claim 26, wherein said disease is a tumor growth, or a cancer.

46. The method of claim 26, wherein said immune response is a cell mediated anti-tumor response.

47. The method of claim 46, wherein said cell mediated response is a CD8+ T cell response.

48. The method of claim 46, wherein said cell mediated response is a CD4+ T cell response.

49. The method of claim 46, wherein said cell mediated response is a natural killer (NK) cell response.

50. The method of any one of claims 26-49, wherein said method delays the onset of or prevents a tumor growth or cancer in a subject.

51. The method of any one of claims 26-49, wherein said method allows delaying the onset of metastatic disease in a subject.

52. The method of any one of claims 26-49, wherein said method results in the breaking of tolerance to a self-antigen in a subject.

53. The method of any one of claims 26-52, wherein said method allows treating a subject having said disease.

54. The method of claim 53, wherein said disease is a tumor growth or cancer.

55. The method of claim 54, further comprising administering a booster of said composition comprising said recombinant Listeria.

56. The method of claim 54, wherein said treating results in progression free survival.

57. The method of claim 54, wherein said treating results in inhibiting tumor growth.

58. The method of claim 54, wherein said treating prolongs survival of said subject having said tumor or cancer.

59. A method of inducing an anti-tumor immune response, said method comprising the step of administering a combination therapy comprising a composition comprising an immunosuppressive antagonist and a composition comprising said recombinant Listeria of any one of claims 1-21.
